(19) United States
(12) Patent Application Publication
(10) Pub. No.: US 2025/0152751 A1
(43) Pub. Date: May 15, 2025

(54) RED-SHIFTED FLUOROPHORES AND METHODS OF USING THE SAME

(71) Applicant: GEORGIA STATE UNIVERSITY RESEARCH FOUNDATION, INC., Atlanta, GA (US)

(72) Inventor: Maged HENARY, Lawrenceville, GA (US)

(21) Appl. No.: 18/834,839

(22) PCT Filed: Feb. 2, 2023
(86) PCT No.: PCT/US2023/012184

§ 371 Date: Jul. 31, 2024

Related U.S. Application Data
(60) Provisional application No. 63/305,780, filed on Feb. 2, 2022.

(51) Int. Cl. A61K 49/00

(52) U.S. Cl. CPC A61K 49/0052

(57) ABSTRACT

The present invention is directed to red-shifted fluorophores, methods of making red-shifted fluorophores, and methods of using red-shifted fluorophores. In some embodiments, the red-shifted fluorophores are cytotoxic, and useful for the treatment of proliferative disorders such as cancer. In some embodiments, the red-shifted fluorophores are useful for imaging and diagnostic applications.

### CROSS-REFERENCE TO RELATED APPLICATIONS

This application claims the benefit of U.S. Provisional Application No. 63/305,780, filed Feb. 2, 2022, which is expressly incorporated herein by reference in its entirety.

### FIELD OF THE INVENTION

The present invention is directed to red-shifted fluorophores, methods of making red-shifted fluorophores, and methods of using red-shifted fluorophores. In some embodiments, the red-shifted fluorophores are cytotoxic, and useful for the treatment of proliferative disorders such as cancer.

### BACKGROUND

Donor-π-acceptor (D-π-A), or red-shifted fluorophores have attracted attention among researchers in the last decade owing to their push-pull systems. This class of fluorophores has a donor unit and an acceptor moiety at each end, which are connected by a conjugated π system. This conjugation enables an intramolecular charge transfer (ICT) and creates the push-pull system, yielding a lower energy molecular orbital (MO). With a combination of strong electron donor and acceptor units at each end of the fluorophore, the fluorescence properties can be altered from ultraviolet-visible (UV-vis) to near infrared (NIR). The NIR window is essential for bioimaging since photons can penetrate deeper into the tissue, and there is low autofluorescence from biomolecules and tissues. In other applications, NIR D-π-A fluorophores can be used as cytotoxic agents for the treatment of cancer and other hyperproliferative disorders. NIR D-π-A fluorophores also find application in various areas such as dye-sensitized solar cells (DSCs), organic light emitting diodes (OLEDs), biosensors, and nonlinear optical imaging.

Most of the D-π-A fluorophores which were reported so far have emission in the UV-vis region. Several studies were performed to synthesize and investigate the optical properties of bathochromic (red) shifted D-π-A fluorophores. In these studies, the effect of various donor and acceptor groups and the effect of the chain length of their π system on absorption and fluorescence were investigated. Factors affecting the properties of D-π-A systems are (1) the strength of donor and acceptor units, (2) the length and the composition of the π linker system, and (3) planarization degree of the fluorophore molecule. These factors may result in a decreased energy band gap between donor and acceptor units, which contributes to the red shift of the emission wavelength maxima.

Some examples of donor units are phenyl, dimethyl-amino, diphenylamino, indoline, carbazole, and julolidine, and examples of commonly used acceptor moieties are trifluoromethyl, cyanoacetic acid, 2-dicyanomethylidene-3-cyano-4,5,5-trimethyl-2,5-dihydrofuran (TCF), pentafluorosul-fonyl, thiobarbituric acid and malononitrole. The π linker between donor and acceptor units also contributes to the optoelectronic properties of the fluorophore since it changes the spatial arrangement and electronic structure of the molecules.

There remains a need for improved NIR D-π-A fluorophores. There remains a need for improved cytotoxic agents for the treatment of proliferative disorders. There remains a need for improved D-π-A fluorophores with improved NIR absorbance.

In accordance with the purposes of the disclosed materials and methods, as embodied and broadly described herein, the disclosed subject matter, in one aspect, relates to compounds, compositions and methods of making and using compounds and compositions.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

The details of one or more embodiments are set forth in the descriptions below. Other features, objects, and advantages will be apparent from the description and from the claims.

### BRIEF DESCRIPTION OF THE FIGURES

FIG. 1A and FIG. 1B depict optical properties of Compound 47.

FIG. 2 depicts photothermal stability for compounds 70-76 relative to ICG.

FIG. 3 depicts photothermal stability for compounds 77-79 relative to ICG.

FIG. 4 depicts photothermal stability for compounds 80-85 relative to ICG.

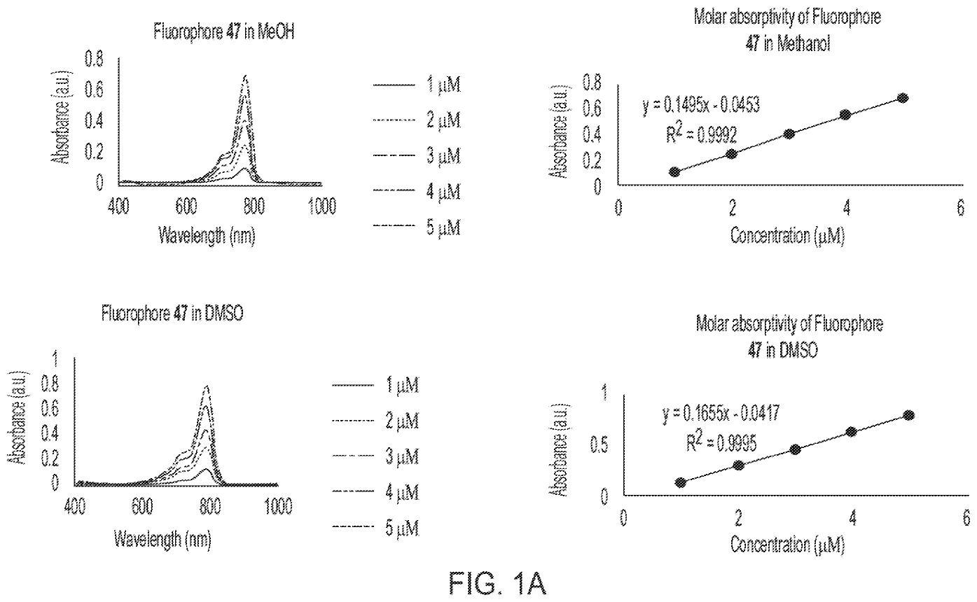

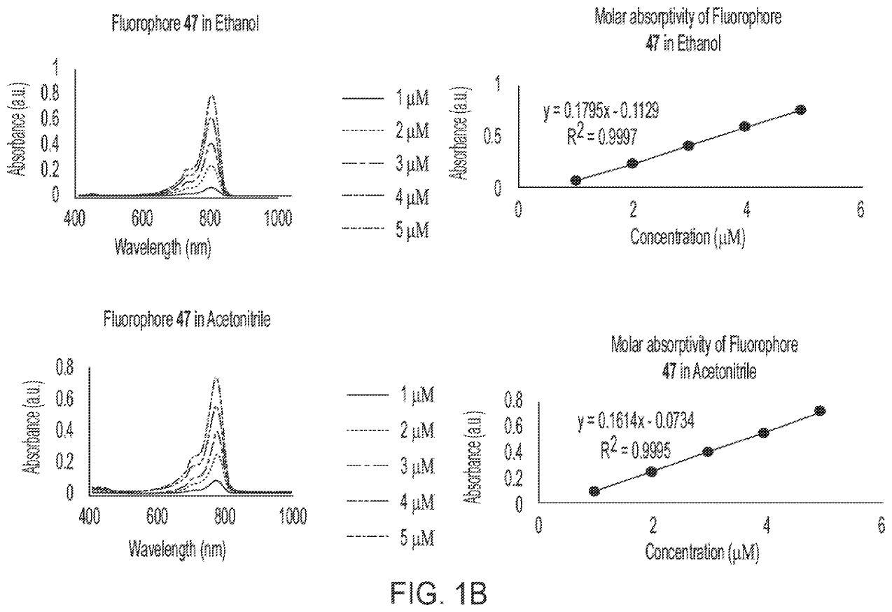

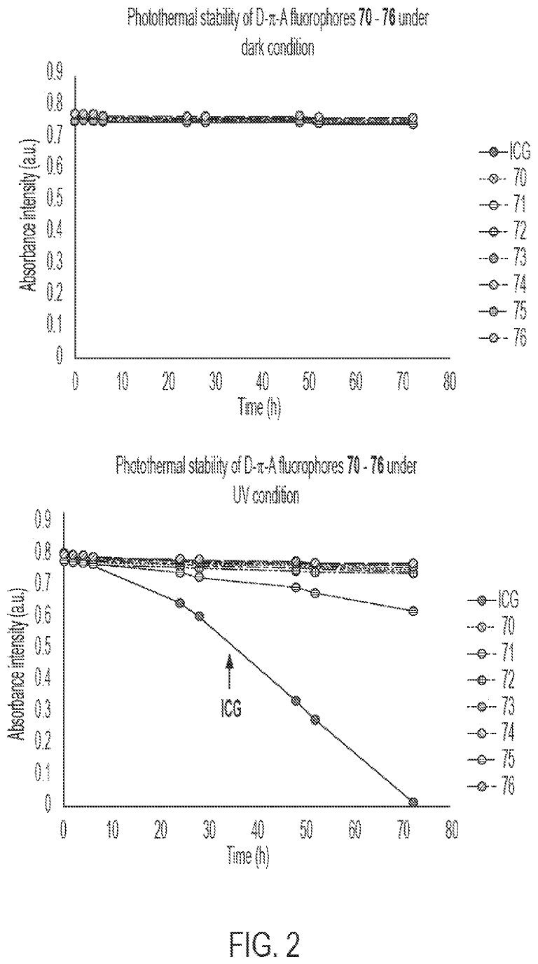

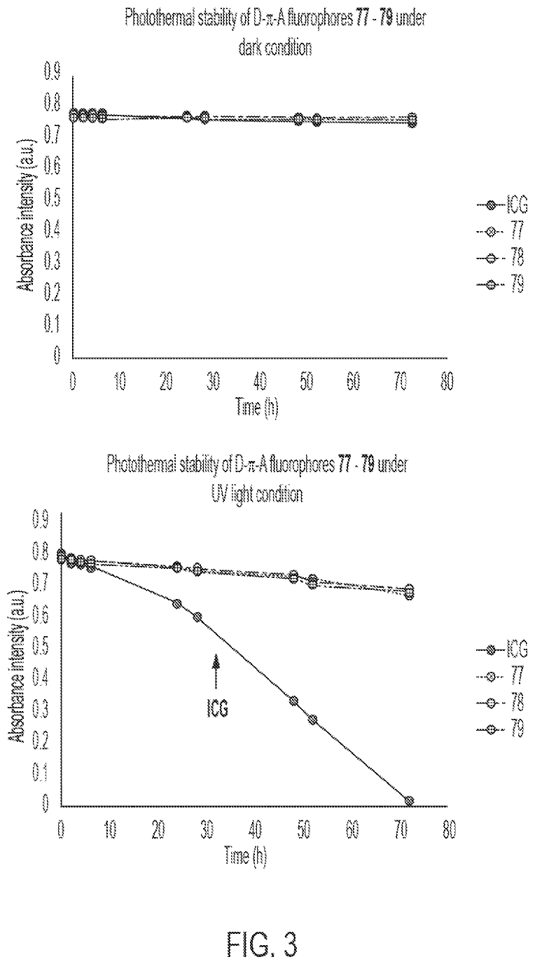

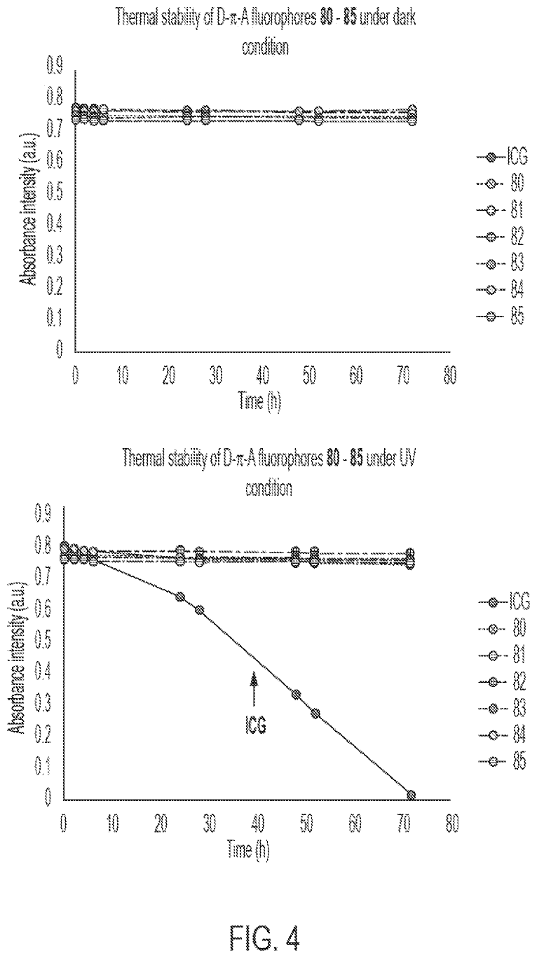

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched hydrocarbon group having a specified range of carbon atoms (e.g., a “C₁₋₁₆ alkyl” can have from 1 to 16 carbon atoms). Unless specified to the contrary, an “alkyl” group includes both saturated alkyl groups and unsaturated alkyl groups. A saturated alkyl group does not include any carbon-carbon double bonds or carbon-carbon triple bonds. An unsaturated alkyl group contains at least one double or triple carbon-carbon bond. In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ saturated alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “alkylenyl” refers to a divalent radical of a straight-chain, cyclic, or branched saturated hydrocarbon group having a specified range of carbon atoms (e.g., a “C₁₋₁₆ alkyl” can have from 1 to 16 carbon atoms). An example of alkylenyl is a methylene (—CH₂—). An alkylenyl can be substituted as described above for an alkyl.

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “hydroxyalkyl” is a substituted alkyl group, wherein one or more of the hydrogen atoms are independently replaced by a hydroxyl. In some embodiments, the hydroxyalkyl moiety has 1 to 8 carbon atoms (“C₁₋₈ hydroxyalkyl”). In some embodiments, the hydroxyalkyl moiety has 1 to 6 carbon atoms (“C₁₋₆ hydroxyalkyl”). In some embodiments, the hydroxyalkyl moiety has 1 to 4 carbon atoms (“C₁₋₄ hydroxyalkyl”). In some embodiments, the hydroxyalkyl moiety has 1 to 3 carbon atoms (“C₁₋₃ hydroxyalkyl”). In some embodiments, the hydroxyalkyl moiety has 1 to 2 carbon atoms (“C₁₋₂ hydroxyalkyl”).

The term “alkoxy” refers to an alkyl group, as defined herein, appended to the parent molecular moiety through an oxygen atom. In some embodiments, the alkoxy moiety has 1 to 8 carbon atoms (“C₁₋₈ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “haloalkoxy” refers to a haloalkyl group, as defined herein, appended to the parent molecular moiety through an oxygen atom. In some embodiments, the alkoxy moiety has 1 to 8 carbon atoms (“C₁₋₈ haloalkoxy”). In some embodiments, the alkoxy moiety has 1 to 6 carbon atoms (“C₁₋₆ haloalkoxy”). In some embodiments, the alkoxy moiety has 1 to 4 carbon atoms (“C₁₋₄ haloalkoxy”). In some embodiments, the alkoxy moiety has 1 to 3 carbon atoms (“C₁₋₃ haloalkoxy”). In some embodiments, the alkoxy moiety has 1 to 2 carbon atoms (“C₁₋₂ haloalkoxy”). Representative examples of haloalkoxy include, but are not limited to, difluoromethoxy, trifluoromethoxy, and 2,2,2-trifluoroethoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋₈ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”).

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and 1 or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

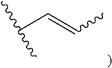

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”).

In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C2_10 alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C_{2_4} alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like.

Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₆). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

As used herein, the term “heterocyclyl” refers to an aromatic (also referred to as a heteroaryl), unsaturated, or saturated cyclic hydrocarbon that includes at least one heteroatom in the cycle. For example, the term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, aziridinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofurany1, tetrahydrothiopheny1, dihydrothiopheny1, pyrrolidiny1, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, lH-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₂, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb} or ═NOR^{cc}; each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion; each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃, —C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb})₂)₂, wherein X⁻, R^{aa}, R^{bb} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb}, and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})², —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₂ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})2, —C(═S)O(R^{X1}), —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di- heteroaliphaticamino, mono- or dialkylamino, mono- or di-heteroalkylamino, mono- or di-arylamino, or mono- or diheteroarylamino; or two R^{X1} groups taken together form a 5- to 6-membered heterocyclic ring.

Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes (CHO), esters (e.g., —CO₂R^{aa}, —C(═O)SR^{aa}, —C(═S)SR^{aa}), amides (e.g., —C(═O)N(R^{bb})₂, C(═O)NR^{bb}SO₂R^{aa}, —C(═S)N(R^{bb})₂, and imines (e.g., —C(═NR^{bb})R^{aa}, —C(═NR^{bb})OR^{aa}), C(═NR^{bb})N(R^{bb})₂, wherein R^{aa} and R^{bb} are as defined herein.

The term “oxo” refers to the group ═O, and the term “thiooxo” refers to the group ═S.

The term “cyano” refers to the group —CN.

The term “azide” refers to the group —N₃.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or a 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined herein.

As used herein, a chemical bond depicted: [symbol] represents either a single, double, or triple bond, valency permitting. By way of example,

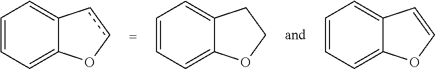

An electron-withdrawing group is a functional group or atom that pulls electron density towards itself, away from other portions of the molecule, e.g., through resonance and/or inductive effects. Exemplary electron-withdrawing groups include F, Cl, Br, I, NO₂, CN, SO₂R, SO₃R, SO₂NR₂, C(O)R^{1a}; C(O)OR, and C(O)NR₂ (wherein R is H or an alkyl, aryl, heteroaryl, cycloalkyl, heterocyclyl group) as well as alkyl group substituted with one or more of those group

An electron-donating group is a functional group or atom that pushes electron density away from itself, towards other portions of the molecule, e.g., through resonance and/or inductive effects. Exemplary electron-donating groups include unsubstituted alkyl or aryl groups, OR and N(R)₂ and alkyl groups substituted with one or more OR and N(R)₂ groups.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Unless stated to the contrary, a substituent drawn without explicitly specifying the point of attachment indicates that the substituent may be attached at any possible atom. For example, in a benzofuran depicted as:

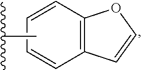

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

As used herein, “control” is an alternative subject or sample used in an experiment for comparison purposes and included to minimize or distinguish the effect of variables other than an independent variable. A “control” can be positive or negative.

As used herein, “therapeutic” generally refers to treating, healing, and/or ameliorating a disease, disorder, condition, or side effect, or to decreasing in the rate of advancement of a disease, disorder, condition, or side effect. The term also includes within its scope enhancing normal physiological function, palliative treatment, and partial remediation of a disease, disorder, condition, side effect, or symptom thereof.

The terms “treating” and “treatment” as used herein refer generally to obtaining a desired pharmacological and/or physiological effect. The effect may be prophylactic in terms of preventing or partially preventing a disease, symptom, or condition thereof.

As used interchangeably herein, “subject,” “individual,” or “patient,” refers to a vertebrate, preferably a mammal, more preferably a human. Mammals include, but are not limited to, murine, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

As used herein, “administration” refers to the injection of active agent on the subject. Exemplary methods of administration include: intravenously (i.v.), intraperitoneally (i.p.), intratumorally (i.t.), or subcutaneously (s.c.) such as tissue ipsilateral (i.l.) to the tumor and tissue contralateral (c.l.) to the tumor.

Disclosed herein are conjugated donor-acceptor fluorophores having the general formula:

[donor]-[linker]-[acceptor],
and pharmaceutically acceptable salts thereof, wherein:
   [donor] has the formula:

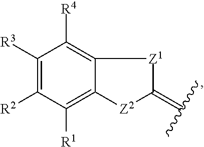

[acceptor] has the formula:

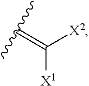

[linker] has the formula:

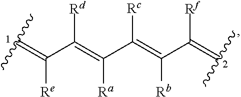

wherein the wavy line marked ‘1’ represents the point of attachment to the [donor] and the wavy line marker ‘2’ represents the point of attachment to the acceptor; and
wherein
Z₂ is N—R^{zd} and Z¹ is O, S, C(R^{z})₂ N—R^{z}, N═CR^{z1}, or C(CR^{z1})═C(CR^{z1}); or
Z² is null and Z¹ is Z³—N(R^{zd})—, wherein Z³ is N═CR^{z3}*, C(CR^{z3})═C(CR^{z3}); O, S, or C(R^{z})₂;
wherein R^{z} and R^{zd} are each case independently optionally substituted C₁₋₆alkyl;
R^{z1} is in case independently selected from F, Cl, Br, I, NO₂, CN, R^{z1}*, OR^{z1}*, N(R^{z1}*)₂, SO₃R^{z1}*, SO₂R^{z1}*, SO₂N(R^{z1}*)₂, C(O)R^{z1}*; C(O)OR^{z1}*, OC(O)R^{z1}*; C(O)N(R^{z1}*)₂, N(R^{z1}*)C(O)R^{z1}*, OC(O)N(R^{z1}*)₂, N(R^{z1}*)C(O)N(R^{z1}*)₂, wherein R^{z1}* is in each case independently selected from hydrogen, C₁₋₈alkyl, aryl, C₁₋₈heteroaryl, C₃₋₈cycloalkyl, or C₁₋₈heterocyclyl;
R^{z3} is in each case independently selected from F, Cl, Br, I, NO₂, CN, R^{z3}*, OR^{z3}*, N(R^{z3}*)₂, SO₃R^{z3}*, SO₂R^{z3}*, SO₂N(R^{z3}*)₂, C(O)R^{z3}*; C(O)OR^{z3}*, OC(O)R^{z3}*; C(O)N(R^{z3}*)₂, N(R^{z3}*)C(O)R^{z3}*, OC(O)N(R^{z3}*)₂, N(R^{z3}*)C(O)N(R^{z3}*)₂, wherein R^{z3}* is in each case independently selected from hydrogen, C₁₋₈alkyl, aryl, C₁₋₈heteroaryl, C₃₋₈cycloalkyl, or C₁₋₈heterocyclyl;
R¹ is F, Cl, Br, I, NO₂, CN, R^{1a}, OR^{1a}, SR^{1a}, N(R^{1a})₂, SO₃R^{1a}, SO₂R^{1a}, SO₂N(R^{1a})₂, C(O)R^{1a}; C(O)OR^{1a}, OC(O)R^{1a}; C(O)N(R^{1a})₂, N(R^{1a})C(O)R^{1a}, OC(O)N(R^{1a})₂, N(R^{1a})C(O)N(R^{1a})₂, wherein R^{1a} is in each case independently selected from hydrogen, C₁₋₈alkyl, aryl, C₁₋₈heteroaryl, C₃₋₈cycloalkyl, or C₁₋₈heterocyclyl;
R² is F, Cl, Br, I, NO₂, CN, R^{2a}, OR^{2a}, SR^{2a}, N(R^{2a})₂, SO₃R^{2a}, SO₂R^{2a}, SO₂N(R^{2a})₂, C(O)R^{2a}; C(O)OR^{2a}, OC(O)R^{2a}; C(O)N(R^{2a})₂, N(R^{2a})C(O)R^{2a}, OC(O)N(R^{2a})₂, N(R^{2a})C(O)N(R^{2a})₂, wherein R^{2a} is in each case independently selected from hydrogen, C₁₋₈alkyl, aryl, C₁₋₈heteroaryl, C₃₋₈cycloalkyl, or C₁₋₈heterocyclyl;
R³ is F, Cl, Br, I, NO₂, CN, R^{3a}, OR^{3a}, SR^{3a}, N(R^{3a})₂, SO₃R^{3a}, SO₂R^{3a}, SO₂N(R^{3a})₂, C(O)R^{3a}; C(O)OR^{3a}, OC(O)R^{3a}; C(O)N(R^{3a})₂, N(R^{3a})C(O)R^{3a}, OC(O)N(R^{3a})₂, N(R^{3a})C(O)N(R^{3a})₂, wherein R^{3a} is in each case independently selected from hydrogen, C₁₋₈alkyl, aryl, C₁₋₈heteroaryl, C₃₋₈cycloalkyl, or C₁₋₈heterocyclyl;
R⁴ is F, Cl, Br, I, NO₂, CN, R^{4a}, OR^{4a}, SR^{4a}, N(R^{4a})₂, SO₃R^{4a}, SO₂R^{4a}, SO₂N(R^{4a})₂, C(O)R^{4a}; C(O)OR^{4a}, OC(O)R^{4a}; C(O)N(R^{4a})₂, N(R^{4a})C(O)R^{4a}, OC(O)N(R^{4a})₂, N(R^{4a})C(O)N(R^{4a})₂, wherein R^{4a} is in each case independently selected from hydrogen, C₁₋₈alkyl, aryl, C₁₋₈heteroaryl, C₃₋₈cycloalkyl, or C₁₋₈heterocyclyl;
X¹ is Cl, F, Br, I, R^{x1}, Q-C(═O)R^{x1}, Q-C(═O)OR^{x1}, Q-C(═O)SR^{x1}, Q-SO₂R^{x1}, Q-SO₃R^{x1}, Q-C(═O)N(R^{x1})₂, Q-C(═NR^{x1′})R^{x1}, Q-C(═NR^{x′})OR^{x1}, Q-SO₂R^{x1}, Q-SO₃R^{x1}, Q-C(NR^{x1′})N(R^{x1})₂, Q-C₁₋₃haloalkyl; Q-CN, Q-NO₂;
X² is Cl, F, Br, I, Q-C(═O)R^{x2}, Q-C(═O)OR^{x2}, Q-C(═O)SR^{x2}, Q-SO₂R^{x2}, Q-SO₃R^{x2}, Q-C(═O)N(R^{x2})₂, Q-C(═NR^{x2′})R^{x2}, Q-C(═NR^{x′})OR^{x2}, Q-SO₂R^{x2}, Q-SO₃R^{x2}, Q-C(NR^{x2′})N(R^{x2})₂, Q-C₁₋₃haloalkyl; Q-CN, Q-NO₂,
wherein Q is in each case independently selected from null, CH═CH, or C≡C;
R^{x1} is in each case independently selected from H, optionally substituted C₁₋₆alkyl, optionally substituted C₁₋₆haloalkyl; optionally substituted C₁₋₁₀aryl; optionally substituted C₃₋₁₀cycloalkyl optionally substituted C₁₋₁₀heteroaryl; optionally substituted C₁₋₁₀heterocyclyl;
R^{x2} is in each case independently selected from H, optionally substituted C₁₋₆alkyl, optionally substituted C₁₋₆haloalkyl; optionally substituted C₁₋₁₀aryl; optionally substituted C₃₋₁₀cycloalkyl optionally substituted C₁₋₁₀heteroaryl; optionally substituted C₁₋₁₀heterocyclyl;
wherein X¹ and X² may together form a ring
R^{a} is F, Cl, Br, I, NO₂, CN, R^{a1}, OR^{a1}, SR^{a1}, N(R^{a1})₂, SO₂R^{a1}, SO₂N(R^{a1})₂, C(O)R^{a1}; C(O)OR^{a1}, OC(O)R^{a1}; C(O)N(R^{a1})₂, N(R^{a1})C(O)R^{a1}, OC(O)N(R^{a1})₂, N(R^{a1})C(O)N(R^{a1})₂, wherein R^{a1} is in each case independently selected from hydrogen, C₁₋₈alkyl, aryl, C₁₋₈heteroaryl, C₃₋₈cycloalkyl, or C₁₋₈heterocyclyl;
R^{b} is F, Cl, Br, I, NO₂, CN, R^{b1}, OR^{b1}, SR^{b1}, N(R^{b1})₂, SO₂R^{b1}, SO₂N(R^{b1})₂, C(O)R^{b1}; C(O)OR^{b1}, OC(O)R^{b1}; C(O)N(R^{b1})₂, N(R^{b1})C(O)R^{b1}, OC(O)N(R^{b1})₂, N(R^{b1})C(O)N(R^{b1})₂, wherein R^{b1} is in each case independently selected from hydrogen, C₁₋₈alkyl, aryl, C₁₋₈heteroaryl, C₃₋₈cycloalkyl, or C₁₋₈heterocyclyl;
R^{c} is F, Cl, Br, I, NO₂, CN, R^{c1}, OR^{c1}, SR^{c1}, N(R^{c1})₂, SO₂R^{c1}, SO₂N(R^{c1})₂, C(O)R^{c1}; C(O)OR^{c1}, OC(O)R^{c1}; C(O)N(R^{c1})₂, N(R^{c1})C(O)R^{c1}, OC(O)N(R^{c1})₂, N(R^{c1})C(O)N(R^{c1})₂, wherein R^{c1} is in each case independently selected from hydrogen, C₁₋₈alkyl, aryl, C₁₋₈heteroaryl, C₃₋₈cycloalkyl, or C₁₋₈heterocyclyl;
R^{d} is F, Cl, Br, I, NO₂, CN, R^{d1}, OR^{d1}, SR^{d1}, N(R^{d1})₂, SO₂R^{d1}, SO₂N(R^{d1})₂, C(O)R^{d1}; C(O)OR^{d1}, OC(O)R^{d1}; C(O)N(R^{d1})₂, N(R^{d1})C(O)R^{d1}, OC(O)N(R^{d1})₂, N(R^{d1})C(O)N(R^{d1})₂, wherein R^{d1} is in each case independently selected from hydrogen, C₁₋₈alkyl, aryl, C₁₋₈heteroaryl, C₃₋₈cycloalkyl, or C₁₋₈heterocyclyl;
R^{e} is F, Cl, Br, I, NO₂, CN, R^{e1}, OR^{e1}, SR^{e1}, N(R^{e1})₂, SO₂R^{e1}, SO₂N(R^{e1})₂, C(O)R^{e1}; C(O)OR^{e1}, OC(O)R^{e1}; C(O)N(R^{e1})₂, N(R^{e1})C(O)R^{e1}, OC(O)N(R^{e1})₂, N(R^{e1})C(O)N(R^{e1})₂, wherein R^{e1} is in each case independently selected from hydrogen, C₁₋₈alkyl, aryl, C₁₋₈heteroaryl, C₃₋₈cycloalkyl, or C₁₋₈heterocyclyl;
R^{f} is F, Cl, Br, I, NO₂, CN, R^{f1}, OR^{f1}, SR^{f1}, N(R^{f1})₂, SO₂R^{f1}, SO₂N(R^{f1})₂, C(O)R^{f1}; C(O)OR^{f1}, OC(O)R^{f1}; C(O)N(R^{f1})₂, N(R^{f1})C(O)R^{f1}, OC(O)N(R^{f1})₂, N(R^{f1})C(O)N(R^{f1})₂, wherein R^{f1} is in each case independently selected from hydrogen, C₁₋₈alkyl, aryl, C₁₋₈heteroaryl, C₃₋₈cycloalkyl, or C₁₋₈heterocyclyl;
wherein any two or more of R¹, R², R³, R⁴, R^{a}, R^{b}, R^{c}, R^{d}, R^{e}, R^{f}, R^{z}, R^{zd}, R^{z1}, R^{z3}, R^{c}, R^{b}, R^{x1}, and R^{x2} may together form a ring.

The skilled person understands the [donor]-[linker]-[acceptor] defined above may be depicted as:

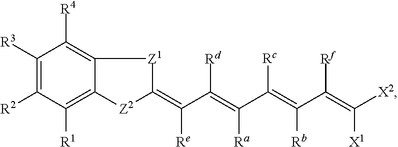

wherein each of the R, Z, and X groups have the meaning given above.

The skilled person appreciates it can be difficult to establish the exact geometric configuration of the acceptor olefin. As such, the depiction of a red-shifted fluorophore having one geometric configuration at the acceptor olefin should be understood to also cover the other isomer, as well as mixtures thereof. For example, a compound depicted as

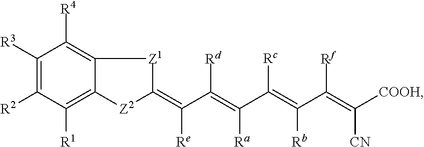

should also be understood to cover the isomer having the formula:

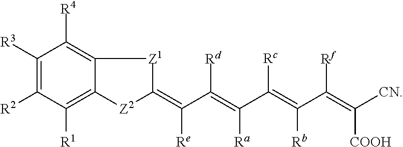

In certain preferred embodiments, R^{c} is R^{c1}, OR^{c1}, SR^{c1}, N(R^{c1})₂, wherein R^{c1} is in each case independently selected from hydrogen, C₁₋₈alkyl, aryl, C₁₋₈heteroaryl, C₃₋₈cycloalkyl, or C₁₋₈heterocyclyl. In some preferred embodiments, R^{c} is N(R^{c1})₂, wherein the two R^{c1} groups together form a ring, optionally substituted one or more times by OH, CH₂OH, COOH, CH₂COOH, NH₂, NHCH₃, N(CH₃)₂, CH₂NH₂, CH₂NHCH₃, CH₂N(CH₃)₂, CH₂CH₂OH, CH₂CH₂COOH, CH₂CH₂NH₂, CH₂CH₂NHCH₃, or CH₂CH₂N(CH₃)₂. In other embodiments, R^{c} is NHR^{c1}, wherein the R^{c1} is CH₂COOH, CH₂CH₂OH, CH₂CH₂COOH, CH₂CH₂NH₂, CH₂CH₂NHCH₃, or CH₂CH₂N(CH₃)₂.

Preferred R^{z} groups include methyl, ethyl, or where two geminal R^{z} groups together form a ring, e.g., cyclopropyl, cyclobutyl, cyclopentyl, cyclohexyl and the like.

In some embodiments, R¹ and R² in the [Donor] can together form a ring:

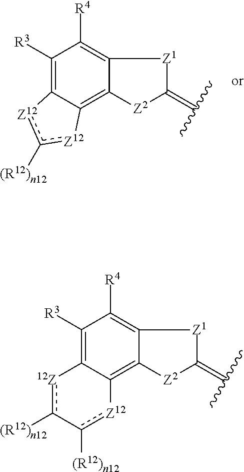

wherein,
R¹² is a substituent defined herein;
n12 is 1 when R¹² is bonded to an sp² hybridized atom and 2 when R¹² is bonded to an sp³ hybridized atom; wherein two geminal R¹² groups can together form an oxo or a thiooxo; Z¹² is in case independently selected from CR¹², C(R¹²)₂, O, S, N, or NH. The skilled person understands that such compounds have the formula:

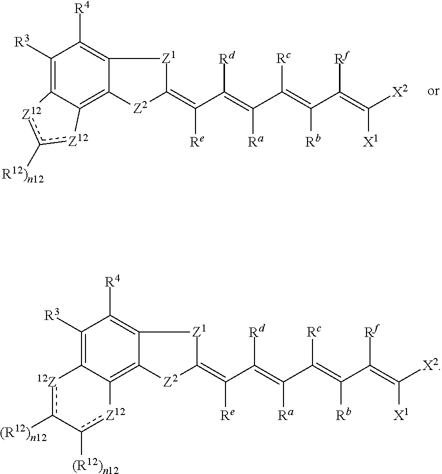

In some embodiments, R² and R³ in the [Donor] can together form a ring

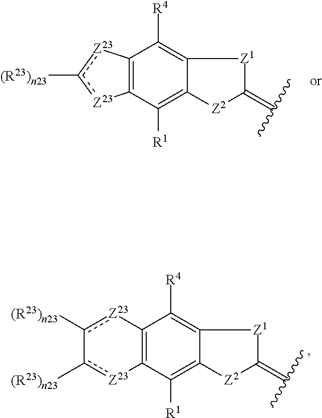

wherein
R²³ is a substituent defined herein;
n23 is 1 when R²³ is bonded to an sp² hybridized atom and 2 when R²³ is bonded to an sp³ hybridized atom; wherein two geminal R²³ groups can together form an oxo or thiooxo;
Z²³ is in case independently selected from CR²³, C(R²³)₂, O, S, N, or NH. The skilled person understands such compounds have the formula:

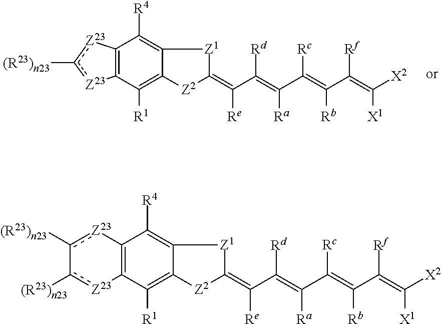

In some embodiments, R³ and R⁴ in the [Donor] can together form a ring:

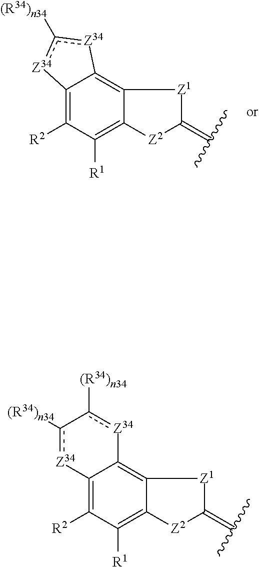

wherein
R³⁴ is a substituent defined herein;
n34 is 1 when R³⁴ is bonded to an sp² hybridized atom and 2 when R³⁴ is bonded to an sp³ hybridized atom; wherein two geminal R³⁴ groups can together form an oxo or a thiooxo; Z³⁴ is in case independently selected from CR³⁴, C(R³⁴)₂, O, S, N, or NH. The skilled person understands that such compounds have the formula:

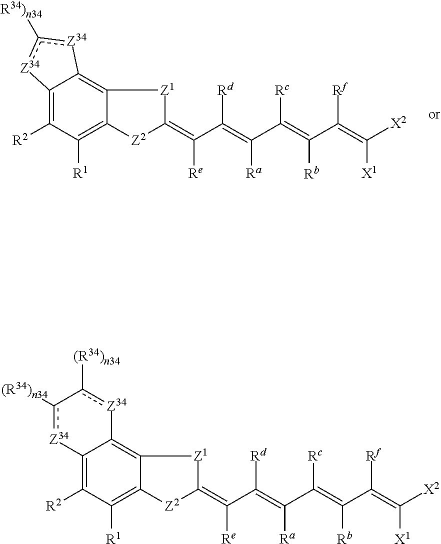

In some instances, Z² can be N—R^{zd}:

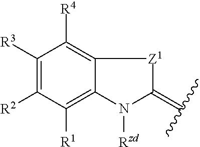

The skilled person understands such a compound has formula:

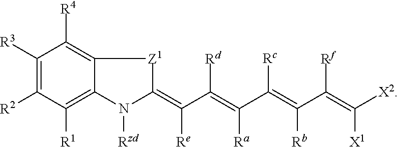

In certain embodiments when Z² is NR^{zd}, R^{zd} and R¹ may together form a ring:

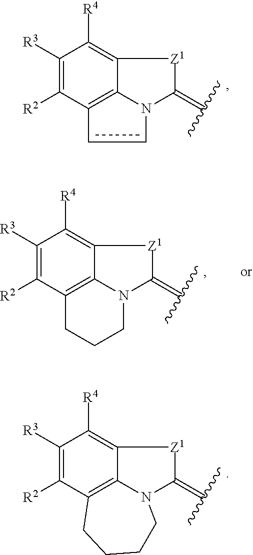

Exemplary systems include:

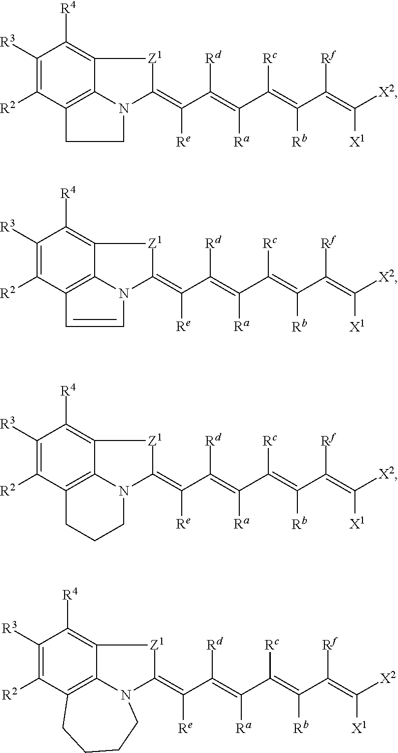

Such rings may be substituted one or more times as defined herein.

In some embodiments where Z² is N—R^{zd}, Z¹ can be O, S, C(R^{z})₂ N—R^{z}, N═CR^{z1}, or C(CR^{z1})═C(CR^{z1}), e.g., the donor can have the formula:

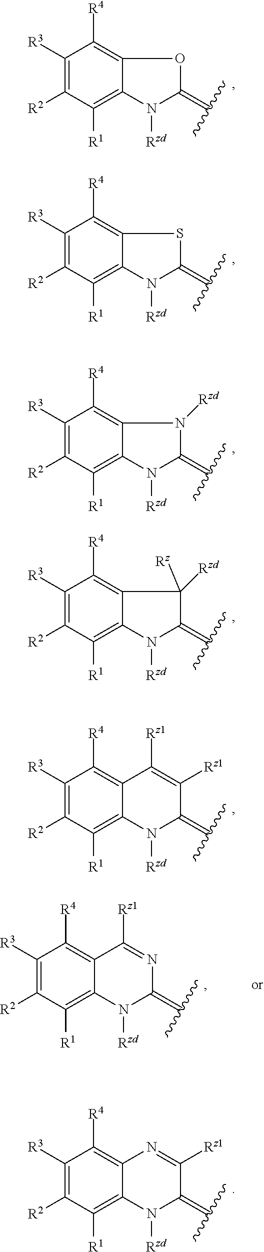

The skilled person understands such compounds have the formula:

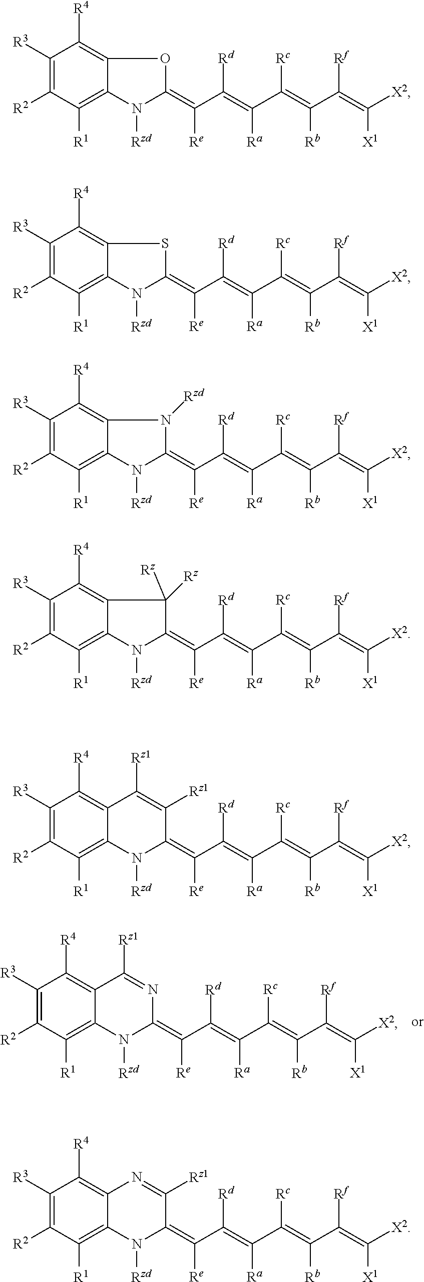

In some embodiments, Z² is null and Z¹ is Z³—N(R^{zd})—:

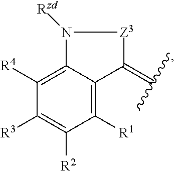

The skilled person understands such compounds have the formula:

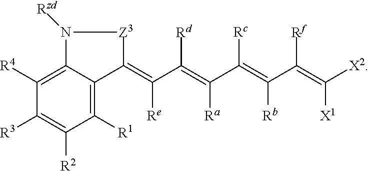

Exemplary Z³ groups include N═CR^{z1}, or C(CR^{z1})═C(CR^{z1}), O, S, or C(R^{z})₂. In some instances when Z² is null and Z¹ is Z³—N(R^{zd})— the [donor] may have the formula:

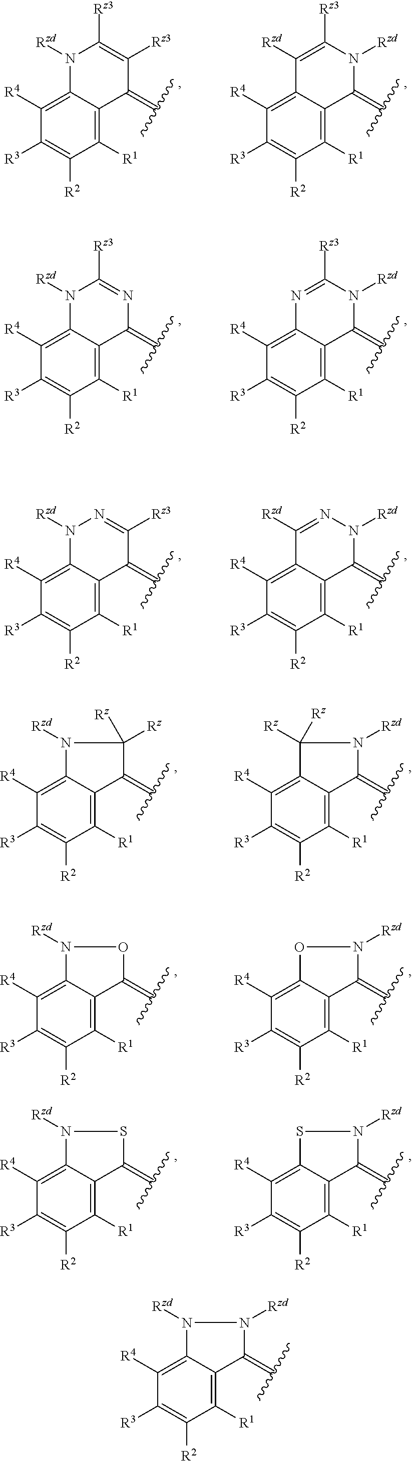

The skilled person understands that such compounds can have the formula:

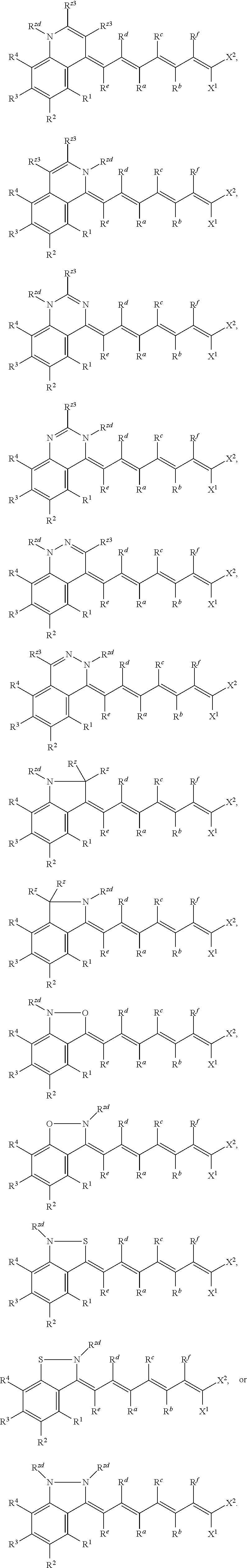

In some embodiments, R^{zd} and R⁴ in the [donor] may together form a ring:

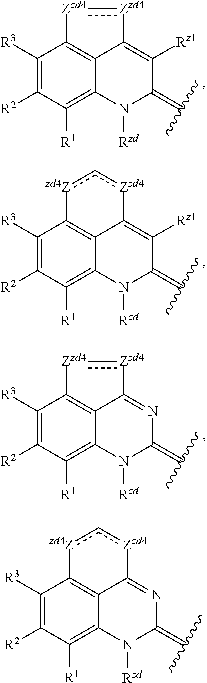

wherein Z^{zd4} is independently selected from CH₂, CH, NR^{zd}, N, O, or S. Examples of such ring systems include:

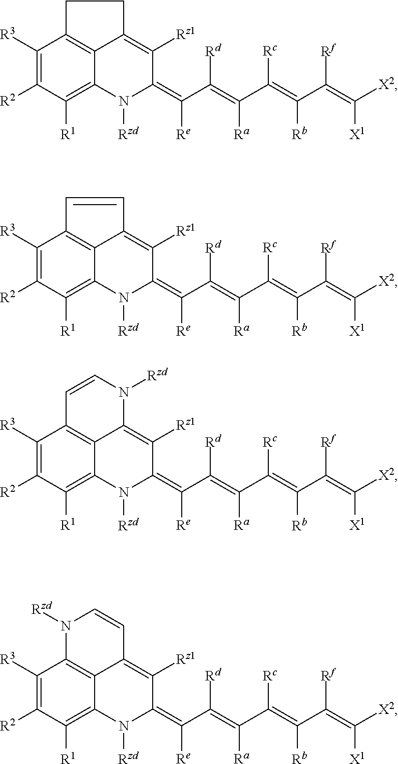

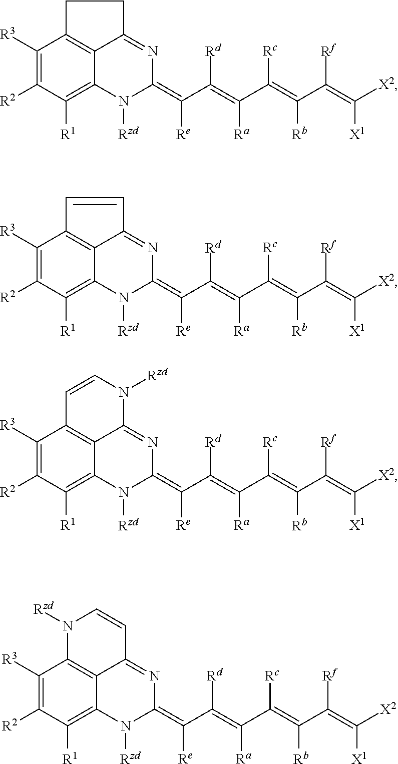

In certain embodiments, the [donor] may be:

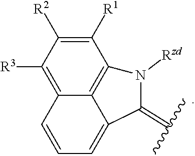

In some embodiments, R^{zd} is C₁₋₈alkyl, optionally substituted one or more times with aryl, OH, NH₂, NHC₁₋₃alkyl, N(C₁₋₃alkyl)₂, N⁺(C₁₋₃alkyl)₃, X⁻, SO₃H, CO₂H, wherein X⁻ is a pharmaceutically acceptable anion. Exemplary R^{zd} groups include methyl, ethyl, benzyl, propyl, n-butyl, n-pentyl, n-hexyl, n-heptyl, n-octyl, 2-hydroxyethyl, 3-hydroxypropyl, 4-hydroxybutyl, 2-aminoethyl, 3-aminopropyl, 4-aminobutyl, 2-phenylethyl, 3-phenylpropyl, 4-phenylbutyl, 2-(dimethylamino)ethyl, 3-(dimethylamino)propyl, 4-(dimethylamino)butyl, 2-(trimethylammonium iodide)ethyl, 3-(trimethylammonium iodide)propyl, 4-(trimethylammonium iodide)butyl, 2-ethanesulfonic acid, 3-propanesulfonic acid, and 4-butanesulfonic acid. The iodide anion may be replaced with another acceptable anion, including bromide, chloride, sulfate, citrate, acetate, ect.

In certain embodiments, one of R¹, R², R³ and R⁴ is an electron-withdrawing group, and the remaining three of R¹, R², R³ and R⁴ are each hydrogen. Exemplary electron-withdrawing groups include F, Cl, Br, I, NO₂, CN, SO₂R^{1a}, SO₂N(R^{1a})₂, C(O)R^{1a}; C(O)OR^{1a}, and C(O)N(R^{1a})₂ (here R^{1a} groups, defined above, are used in the exemplary, not limiting, fashion). In some embodiments the electron-withdrawing group is an alkyl group substituted with one or more of F, Cl, Br, I, NO₂, CN, SO₂R^{1a}, SO₂N(R^{1a})₂, C(O)R^{1a}; C(O)OR^{1a}, and C(O)N(R^{1a})₂. Preferable electron-withdrawing groups include F, Cl, Br, I, C₁₋₃haloalkyl, C(O)C₁₋₃alkyl, C(O)OC₁₋₃alkyl, C(O)NHC₁₋₃alkyl, C(O)OH, SO₃H, C(O)C₁₋₃haloalkyl, C(O)OC₁₋₃haloalkyl, and C(O)NHC₁₋₃haloalkyl.

In some embodiments, R¹ is an electron-withdrawing group as defined herein and each of R², R³, and R⁴ are hydrogen.

In some embodiments, R² is an electron-withdrawing group as defined herein and each of R¹, R³, and R⁴ are hydrogen.

In some embodiments, R³ is an electron-withdrawing group as defined herein and each of R¹, R², and R⁴ are hydrogen.

In some embodiments, R⁴ is an electron-withdrawing group as defined herein and each of R¹, R², and R³ are hydrogen.

In some embodiments, R¹ and R³ are independently electron-withdrawing group as defined herein and each of R², and R⁴ are hydrogen.

In some embodiments, R² and R⁴ are independently electron-withdrawing group as defined herein and each of R¹, and R³ are hydrogen.

In certain embodiments, when Z² is N—R^{zd}, R³ is an electron-withdrawing group as defined herein and each of R¹, R², and R⁴ are hydrogen. In certain embodiments, when Z² is N—R^{zd}, R¹ is an electron-withdrawing group as defined herein and each of R², R³, and R⁴ are hydrogen. In other embodiments, R¹ and R³ are electron-withdrawing groups as defined herein and R² and R⁴ are hydrogen. In certain embodiments, when Z² is N—R^{zd}, R¹ and R³ are electron-withdrawing groups as defined herein and R² and R⁴ are hydrogen.

In certain embodiments, when Z² is null, R² is an electron-withdrawing group as defined herein and each of R², R³, and R⁴ are hydrogen. In certain embodiments, when Z² is null, R⁴ is an electron-withdrawing group as defined herein and each of R¹, R², and R³ are hydrogen. In other embodiments, R² and R⁴ are electron-withdrawing groups as defined herein and R¹ and R³ are hydrogen. In certain embodiments, when Z² is null, R² and R⁴ are electron-withdrawing groups as defined herein and R¹ and R³ are hydrogen.

In other embodiment, one of R¹, R², R³ and R⁴ is an electron-donating group, and the remaining three of R¹, R², R³ and R⁴ are each hydrogen. Exemplary electron-donating groups include OR^{1a} and N(R^{1a})₂ (here R^{1a} groups, defined above, are used in the exemplary, not limiting, fashion). Preferable electron-donating groups include OH, NH₂, OC₁₋₃alkyl, NHC₁₋₃alkyl, N(C₁₋₃alkyl)₂, OC₁₋₃alkyl, NHC₁₋₃alkyl, and N(C₁₋₃alkyl)₂, as well as unsubstituted alkyl and aryl groups, and alkyl and aryl groups substituted with one or more of OH, NH₂, OC₁₋₃alkyl, NHC₁₋₃alkyl, N(C₁₋₃alkyl)₂, OC₁₋₃alkyl, NHC₁₋₃alkyl, and N(C₁₋₃alkyl)₂.

In some embodiments, R¹ is an electron-donating group as defined herein and each of R², R³, and R⁴ are hydrogen.

In some embodiments, R² is an electron-donating group as defined herein and each of R¹, R³, and R⁴ are hydrogen.

In some embodiments, R³ is an electron-donating group as defined herein and each of R¹, R², and R⁴ are hydrogen.

In some embodiments, R⁴ is an electron-donating group as defined herein and each of R¹, R², and R³ are hydrogen.

In some embodiments, R¹ and R³ are independently electron-donating group as defined herein and each of R², and R⁴ are hydrogen.

In some embodiments, R² and R⁴ are independently electron-donating group as defined herein and each of R¹, and R³ are hydrogen.

In certain embodiments, when Z² is N—R^{zd}, R³ is an electron-donating group as defined herein and each of R¹, R², and R⁴ are hydrogen. In certain embodiments, when Z² is N—R^{zd}, R¹ is an electron-donating group as defined herein and each of R², R³, and R⁴ are hydrogen. In other embodiments, R¹ and R³ are electron-donating groups as defined herein and R² and R⁴ are hydrogen. In certain embodiments, when Z² is N—R^{zd}, R¹ and R³ are electron-donating groups as defined herein and R² and R⁴ are hydrogen.

In certain embodiments, when Z² is null, R² is an electron-donating group as defined herein and each of R², R³, and R⁴ are hydrogen. In certain embodiments, when Z² is null, R⁴ is an electron-donating group as defined herein and each of R¹, R², and R³ are hydrogen. In other embodiments, R² and R⁴ are electron-donating groups as defined herein and R¹ and R³ are hydrogen. In certain embodiments, when Z² is null, R² and R⁴ are electron-donating groups as defined herein and R¹ and R³ are hydrogen.

In certain embodiments, each of R^{a}, R^{b}, R^{d}, R^{e}, and R^{f} in the [linker] are hydrogen.

In other embodiments, R^{e} is an electron-withdrawing or electron-donating group and R^{d} and R^{f} are each hydrogen. In some preferred embodiments R^{e} is an electron-donating group and R^{d} and R^{f} are each hydrogen. In other embodiments, R^{f} is an electron-withdrawing or electron-donating group and R^{d} and R^{e} are each hydrogen. In some preferred embodiments, R^{f} is an electron-withdrawing group and R^{d} and R^{e} are each hydrogen.

Preferable electron-donating groups for R^{d}, R^{e}, and R^{f} include OH, NH₂, OC₁₋₃alkyl, NHC₁₋₃alkyl, N(C₁₋₃alkyl)₂, OC₁₋₃alkyl, NHC₁₋₃alkyl, and N(C₁₋₃alkyl)₂, as well as unsubstituted alkyl and aryl groups, and alkyl and aryl groups substituted with one or more of OH, NH₂, OC₁₋₃alkyl, NHC₁₋₃alkyl, N(C₁₋₃alkyl)₂, OC₁₋₃alkyl, NHC₁₋₃alkyl, and N(C₁₋₃alkyl)₂.

Preferable electron-withdrawing groups for R^{d}, R^{e}, and R^{f} include F, Cl, Br, I, C₁₋₃haloalkyl, C(O)C₁₋₃alkyl, C(O)OC₁₋₃alkyl, C(O)NHC₁₋₃alkyl, C(O)OH, SO₃H, C(O)C₁₋₃haloalkyl, C(O)OC₁₋₃haloalkyl, and C(O)NHC₁₋₃haloalkyl.

In some embodiments R^{e} is an electron-donating group and R^{f} is an electron-withdrawing group.

In some preferred embodiments R^{a} and R^{b} together form a ring, e.g., the [linker] has the formula:

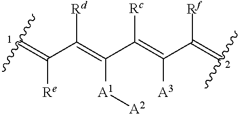

wherein:
A¹ is selected from C(R^{a1})₂, N(R^{a1}*)ₙₐ₁, O, S, —C(R^{a1})₂—C(R^{a1})₂—, —C(R^{a1})═C(R^{a1})—, —C(R^{a1})═N—;
R^{a1} is selected from F, Cl, Br, I, NO₂, CN, R^{a1$}, OR^{a1$}, N(R^{a1$})₂, SO₂R^{a1$}, SO₂N(R^{a1$})₂, C(O)R^{a1$}; C(O)OR^{a1$}, OC(O)R^{a1$}; C(O)N(R^{a1$})₂, N(R^{a1$})C(O)R^{a1$}, OC(O)N(R^{a1$})₂, N(R^{a1$})C(O)N(R^{a1$})₂, wherein R^{a1$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
na1 is 0, 1 or 2;
when na1 is 1 then R^{a1}* is selected from R^{a1$}, SO₂R^{a1$}, SO₂N(R^{a1$})₂, C(O)R^{a1$}; C(O)OR^{a1$}, C(O)N(R^{a1$})₂, N(R^{a1$})C(O)R^{a1$}, wherein R^{a1$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
when na1 is 2 then R^{a1}* is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
A² is selected from null, C(R^{a2})₂, N(R^{a2}*)ₙₐ₂, O, S, —C(R^{a2})₂—C(R^{a2})₂—, —C(R^{a2})═C(R^{a2})—, —C(R^{a2})═N—;
R^{a2} is selected from F, Cl, Br, I, NO₂, CN, R^{a2$}, SR^{a2$}, SeR^{a2$}, OR^{a2$}, N(R^{a2$})₂, SO₂R^{a2$}, SO₂N(R^{a2$})₂, C(O)R^{a2$}; C(O)OR^{a2$}, OC(O)R^{a2$}; C(O)N(R^{a2$})₂, N(R^{a2$})C(O)R^{a2$}, OC(O)N(R^{a2$})₂, N(R^{a2$})C(O)N(R^{a2$})₂, wherein R^{a2$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
na2 is 0, 1 or 2;
when na2 is 1 then R^{a2}* is selected from R^{a2$}, SO₂R^{a2$}, SO₂N(R^{a2$})₂, C(O)R^{a2$}; C(O)OR^{a2$}, C(O)N(R^{a2$})₂, N(R^{a2$})C(O)R^{a2$}, wherein R^{a2$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
when na2 is 2 then R^{a2}* is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
A³ is selected from C(R^{a3})₂, N(R^{a3}*)ₙₐ₃, O, S, —C(R^{a3})₂—C(R^{a3})₂—, —C(R^{a3})═C(R^{a3})—, —C(R^{a3})═N—, R^{a3} is selected from F, Cl, Br, I, NO₂, CN, R^{a3$}, OR^{a3$}, N(R^{a3$})₂, SO₂R^{a3$}, SO₂N(R^{a3$})₂, C(O)R^{a3$}; C(O)OR^{a3$}, OC(O)R^{a3$}; C(O)N(R^{a3$})₂, N(R^{a3$})C(O)R^{a3$}, OC(O)N(R^{a3$})₂, N(R^{a3$})C(O)N(R^{a3$})₂, wherein R^{a3$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
na3 is 0, 1 or 2;
when na3 is 1 then R^{a3}* is selected from R^{a3$}, SO₂R^{a3$}, SO₂N(R^{a3$})₂, C(O)R^{a3$}; C(O)OR^{a3$}, C(O)N(R^{a3$})₂, N(R^{a3$})C(O)R^{a3$}, wherein R^{a3$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
when na3 is 2 then R^{a3}* is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl.

In some embodiments, the [linker] has the formula

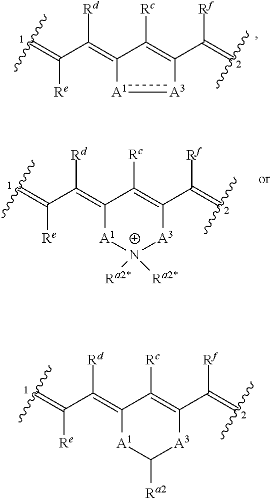

wherein R^{a2}* is in each case independently selected from C₁₋₄alkyl, benzyl phenyl, or H. In the case of the quaternary ammonium ion, the compound will be electronically neutral by virtue of a separate, pharmaceutically acceptable anion (e.g., iodide, bromide, chloride, acetate, sulfate, etc), or from an anionic group (e.g., sulfonate, carboxylate, phosphonate, etc) somewhere else within the molecule. When A² is N⁺(R^{a2}*)₂, R^{a2}* can in each case be independently selected from methyl, ethyl, n-propyl, n-butyl, and benzyl. When A² is CHR^{a2}*, R^{a2}* can be H, methyl, ethyl, n-propyl, n-butyl, isopropyl, tert-butyl, phenyl, and benzyl.

In certain instances when A² is CHR^{a2} O, S, NR^{a2}* or N⁺(R^{a2}*)₂, A¹ can be CH₂ or CH₂CH₂, preferably CH₂ and A³ is CH₂ or CH₂CH₂, preferably CH₂.

In other instances, A² is null, and the [linker] has the formula:

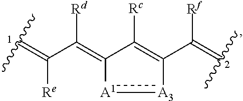

In certain instances when A² is null, A¹ can be CR^{a1}* or C(R^{a1}*)₂, depending on valence, and A³ can be CR^{a3}* or C(R^{a3}*)₂, depending on valence. The skilled person understands that when A¹ is CR^{a1}* (i.e., a sp² hybridized carbon atom) then A³ is CR^{a3}*. Likewise, when A¹ is C(R^{a1}*)₂ (i.e., a sp³ hybridized carbon atom) then A³ is C(R^{a3}*)₂. In other instances, A¹ can be N and A³ can be CR^{a3}*, or A¹ can be CR^{a1}* and A³ can be N. In further embodiments, In other instances, A¹ can be NR^{a1}* and A³ can be C(R^{a3}*)₂, or A¹ can be C(R^{a1}*)₂ and A³ can be NR^{a3}*.

In some instances, the [linker] has the formula:

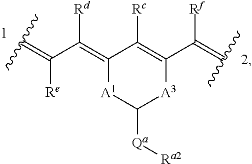

wherein Q^{a} is null, CH₂, CH₂CH₂, or CH₂CH₂CH₂, and R² is H, CH₃, CF₃, CH₂F, CHF₂, CH₂CH₃, CH(CH₃)₂, C(CH₃)₃, F, Cl, Br, SH, SeH, OH, NH₂, SO₃H, PO₃H₂, COOH, SCH₃, SCH₂CH₃, SCH(CH₃)₂, SeCH₃, SeCH₂CH₃, SeCH(CH₃)₂, OCH₃, OCH₂CH₃, OCH(CH₃)₂, OCF₃, OCH₂CF₃, NHCH₃, N(CH₃)₂, ϕ, CH₂CH₃, CH₂CF₃, CH₂CH₂F, CH₂CHF₂, CH(CH₃)₂, C(CH₃)₃, CH₂OH, CH₂NH₂, CH₂SO₃H, CH₂PO₃H₂, CH₂COOH, CH₂OCH₃, CH₂OCF₃, CH₂OCH₂CF₃, CH₂NHCH₃, CH₂N(CH₃)₂, CH₂ϕ, CH₂CH₂CH₃, CH₂CH₂CF₃, CH₂CH₂CH₂F, CH₂CH₂CHF₂, CH₂CH(CH₃)₂, CH₂C(CH₃)₃, CH₂CH₂OH, CH₂CH₂NH₂, CH₂CH₂SO₃H, CH₂CH₂PO₃H₂, CH₂CH₂OOH, CH₂CH₂OCH₃, CH₂CH₂OCF₃, CH₂CH₂OCH₂CF₃, CH₂CH₂NHCH₃, CH₂CH₂N(CH₃)₂, CH₂CH₂ϕ, SCH₂ϕ, SCH₂CH₂CH₃, SCH₂CH₂CF₃, SCH₂CH₂CH₂F, SCH₂CH₂CHF₂, SCH₂CH(CH₃)₂, SCH₂C(CH₃)₃, SCH₂CH₂OH, SCH₂CH₂NH₂, SCH₂CH₂SO₃H, SCH₂CH₂PO₃H₂, SCH₂CH₂COOH, SCH₂CH₂OCH₃, SCH₂CH₂OCF₃, SCH₂CH₂OCH₂CF₃, SCH₂CH₂NHCH₃, SCH₂CH₂N(CH₃)₂, SCH₂CH₂ϕ, OCH₂ϕ, OCH₂CH₂CH₃, OCH₂CH₂CF₃, OCH₂CH₂CH₂F, OCH₂CH₂CHF₂, OCH₂CH(CH₃)₂, OCH₂C(CH₃)₃, OCH₂CH₂OH, OCH₂CH₂NH₂, OCH₂CH₂OO₃H, OCH₂CH₂PO₃H₂, OCH₂CH₂COOH, OCH₂CH₂OCH₃, OCH₂CH₂OCF₃, OCH₂CH₂OCH₂CF₃, OCH₂CH₂NHCH₃, OCH₂CH₂N(CH₃)₂, OCH₂CH₂ϕ, SeCH₂ϕ, SeCH₂CH₂CH₃, SeCH₂CH₂CF₃, SeCH₂CH₂CH2F, SeCH₂CH₂CHF₂, SeCH₂CH(CH₃)₂, SeCH₂C(CH₃)₃, SeCH₂CH₂OH, SeCH₂CH₂NH₂, SeCH₂CH₂SEO₃H, SeCH₂CH₂PO₃H₂, SeCH₂CH₂COOH, SeCH₂CH₂OCH₃, SeCH₂CH₂OCF₃, SeCH₂CH₂OCH₂CF₃, SeCH₂CH₂NHCH₃, SeCH₂CH₂N(CH₃)₂, SeCH₂CH₂ϕ,
wherein ϕ is an aryl or heteroaryl group.

In some instances ϕ is an aryl or heteroaryl having the formula:

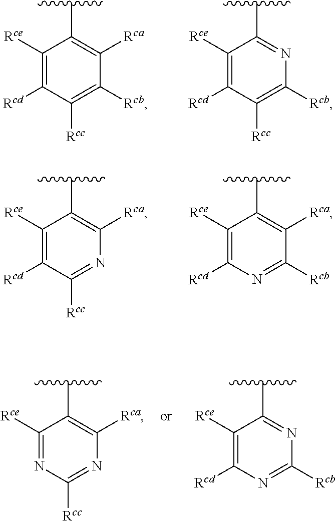

wherein
R^{ca} is selected from H, C₁₋₃alkyl, C₁₋₃haloalkyl, C₁₋₃alkoxyl, F, Cl, Br, OH, NH₂, NHCH₃, N(CH₃)₂, or COOH,
R^{cb} is selected from H, C₁₋₃alkyl, C₁₋₃haloalkyl, C₁₋₃alkoxyl, F, Cl, Br, OH, NH₂, NHCH₃, N(CH₃)₂, or COOH,
R^{cc} is selected from H, C₁₋₃alkyl, C₁₋₃haloalkyl, C₁₋₃alkoxyl, F, Cl, Br, OH, NH₂, NHCH₃, N(CH₃)₂, or COOH,
R^{cd} is selected from H, C₁₋₃alkyl, C₁₋₃haloalkyl, C₁₋₃alkoxyl, F, Cl, Br, OH, NH₂, NHCH₃, N(CH₃)₂, or COOH, and
R^{ce} is selected from H, C₁₋₃alkyl, C₁₋₃haloalkyl, C₁₋₃alkoxyl, F, Cl, Br, OH, NH₂, NHCH₃, N(CH₃)₂, or COOH.

In preferred embodiments Q^{a} is null or CH₂.

In certain embodiments, R^{c} can be F, Cl, Br, I, C₁₋₈alkyl, C₃₋₈cycloalkyl, C₃₋₈cycloalkyl, aryl, heteroaryl, OR^{c1}, SR^{2a}, or NR^{c1}R^{c2}, wherein
R^{c1} is selected from H, C₁₋₈alkyl, C₃₋₈cycloalkyl, C₁₋₈alkyl-OH, C₁₋₈alkyl-COOH, C₁₋₈alkyl-NH₂, C₁₋₈alkyl-NHCH₃, or C₁₋₈alkyl-N(CH₃)₂, and
R^{c2} is selected from H, C₁₋₈alkyl, C₃₋₈cycloalkyl, C₁₋₈alkyl-OH, C₁₋₈alkyl-COOH, C₁₋₈alkyl-NH₂, C₁₋₈alkyl-NHCH₃, or C₁₋₈alkyl-N(CH₃)₂,
wherein R^{c1} and R^{c2} can together form a ring.

In certain preferred embodiments, R^{c} is F, Cl, Br or I.

In some embodiments, R^{c} is SR^{c1}, wherein R^{c1} is selected from H, CH₃, CH₂CH₃, CH(CH₃)₂, CH₂CH₂OH, CH₂COOH, CH₂CH₂COOH.

In some embodiments, R^{c} is OR^{c1}, wherein R^{c1} is selected from H, CH₃, CH₂CH₃, CH(CH₃)₂, CH₂CH₂OH, CH₂COOH, CH₂CH₂COOH. =

In some embodiments, R^{c} is NR^{c1}R^{c2}, wherein R^{c1} and R^{c2} are different or the same, and are selected from H, CH₃, CH₂CH₃, CH(CH₃)₂, CH₂CH₂OH, CH₂COOH, CH₂CH₂COOH. In certain preferred embodiments, R^{c1} and R^{c2} are the same, and are selected from H, CH₃, CH₂CH₃, CH(CH₃)₂, CH₂CH₂OH, CH₂COOH, CH₂CH₂COOH.

In certain embodiments, R^{c} is NR^{c1}R^{c2}, wherein R^{c2} is hydrogen, and R^{c1} is a C₁₋₈alkyl group substituted one or more times by OH, NH₂, COOH, or a combination thereof. By way of example, R^{c1} can be CH₂CH₂OH, CH(COOH)CH2OH, CH₂CH(OH)COOH, CH₂CH₂CH(NH₂)COOH, CH₂CH₂CH₂CH₂CH(NH₂)COOH, or CH₂CH₂CH₂CH₂CH₂COOH.

In other embodiments R^{c} is NR^{c1}R^{c2} and R^{c1} and R^{c2} can together form a heterocyclic ring, for instance a piperidine, pyrrolidine, morpholine, piperazine, which may be further substituted one or more times by F, Cl, Br, I, NO₂, CN, R^{c3}, OR^{c3}, N(R^{c3})₂, SO₂R^{c3}, SO₂N(R^{c3})₂, C(O)R^{c3}; C(O)OR^{c3}, OC(O)R^{c3}; C(O)N(R^{c3})₂, N(R^{c3})C(O)R^{c3}, OC(O)N(R^{c3})₂, N(R^{c3})C(O)N(R^{c3})₂, wherein R^{c3} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl.

In some implementations, R^{c} has the formula:

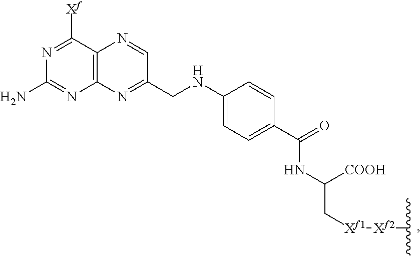

wherein X^{f} is OH or NH₂, X^{f1} is null, phenyl, C₁₋₄alkylene, and X^{f2} is null, O, NH, or S. The skilled person understands various tautomers exist for the pteridine ring (i.e., including one or more carbonyl or imine functional groups). The depiction of one tautomeric form is intended to cover each and every tautomer, either as a mixture or single compound.

In some embodiments, R^{c} has the formula:

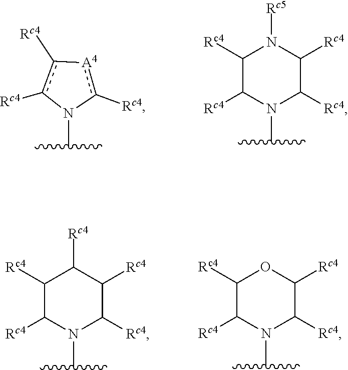

wherein A⁴ is selected from O, S, C(R^{c4})ₙₐ₄, N(R^{c5})ₙₐ₄;
na4 is 0, 1, or 2;
R^{c4} is in each case independently selected from F, Cl, Br, I, NO₂, CN, R^{c4$}, Q⁴-OR^{c4$}, Q⁴-N(R^{c4$})₂, Q⁴-SO₂R^{c4$}, Q⁴-SO₂N(R^{c4$})₂, Q⁴-C(O)R^{c4$}; Q⁴-C(O)OR^{c4$}, Q⁴-OC(O)R^{c4$}; Q⁴-C(O)N(R^{c4$})₂, Q⁴-N(R^{c4$})C(O)R^{c4$}, Q⁴-OC(O)N(R^{c4$})₂, Q⁴-N(R^{c4$})C(O)N(R^{c4$})₂, wherein Q⁴ is null or a C₁₋₄alkylene group, and R^{c4$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl; and
C^{c5} is in each case independently selected from R^{c5$}, Q⁵-OR^{c5$}, Q⁵-N(R^{c5$})₂, Q⁵-SO₂R^{c5$}, Q⁵-SO₂N(R^{c5$})₂, Q⁵-C(O)R^{c5$}; Q⁵-C(O)OR^{c5$}, Q⁵-C(O)N(R^{c5$})₂, wherein Q⁵ is null or a C₁₋₄alkylene group, and R^{c5$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl; wherein any two of more of R^{c4} and R^{c5} can together form a ring.

In some implementations, R^{c5} has the formula:

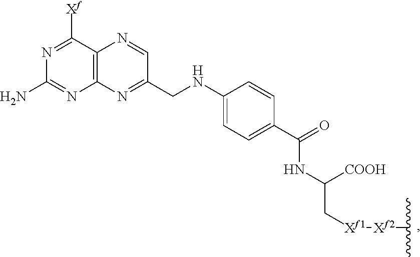

wherein X^{f} is OH or NH₂, X^{f1} is null, phenyl, C₁₋₄alkylene, and X^{f2} is null, O, NH, or S. The skilled person understands various tautomers exist for the pteridine ring (i.e., including one or more carbonyl or imine functional groups). The depiction of one tautomeric form is intended to cover each and every tautomer, either as a mixture or single compound.

In certain preferred embodiments, R^{c} is a group having the formula:

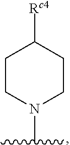

wherein R^{c4} is F, Cl, Br, I, NO₂, CN, R^{c4$}, Q⁴-OR^{c4$}, Q⁴-N(R^{c4$})₂, Q⁴-SO₂R^{c4$}, Q⁴-SO₂N(R^{c4$})₂, Q⁴-C(O)R^{c4$}; Q⁴-C(O)OR^{c4$}, Q⁴-OC(O)R^{c4$}; Q⁴-C(O)N(R^{c4$})₂, Q⁴-N(R^{c4$})C(O)R^{c4$}, Q⁴-OC(O)N(R^{c4$})₂, Q⁴-N(R^{c4$})C(O)N(R^{c4$})₂, wherein Q⁴ is null or a C₁₋₄alkylene group, and R^{c4$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl.

In some implementations, R^{c4} has the formula:

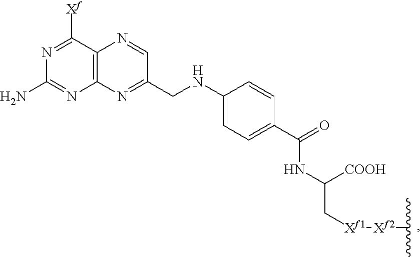

wherein X^{f} is OH or NH₂, X^{f1} is null, phenyl, C₁₋₄alkylene, and X^{f2} is null, O, NH, or S. The skilled person understands various tautomers exist for the pteridine ring (i.e., including one or more carbonyl or imine functional groups). The depiction of one tautomeric form is intended to cover each and every tautomer, either as a mixture or single compound.

In some embodiments when R^{c} is:

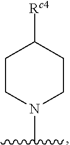

R^{c4} can be R^{c4$}, Q⁴-OR^{c4$}, Q⁴-N(R^{c4$})₂, or Q⁴-C(O)OR^{c4$}, Q⁴ is null, —CH₂—, —CH₂CH₂— or —CH₂CH₂CH₂—, and R^{c4$} is independently H, CH₃, CH₂CH₃, CH(CH₃)₂, or C(CH₃)₃, preferably H.

In some embodiments, one of X¹ or X² is Q²-CO₂H, Q²-CO₂C₁₋₃alkyl, Q²-CO₂C₁₋₃haloalkyl, Q²-SO₂OH, Q²-SO₂OC₁₋₃alkyl, Q²-SO₂OC₁₋₃haloalkyl, Q²-SO₂C₁₋₃alkyl, CSO₂C₁₋₃haloalkyl, Q²-C(O)NHC₁₋₃alkyl, Q²-C(O)NHC₁₋₃haloalkyl, Q²-C(O)NH₂, Q²-C(O)NHC₁₋₃alkyl, Q²-C(O)NHC₁₋₃haloalkyl, wherein Q² is null or —CH═CH—, and the other of X¹ or X² is selected from Cl, F, CN, C₁₋₃haloalkyl, e.g., trifluoromethyl, difluoromethyl, fluoromethyl, 2,2,2-trifluoroethyl, and the like.

In some implementations, both of X¹ and X² are CN.

In some embodiments, X¹ and X² can together form a ring in the [acceptor]:

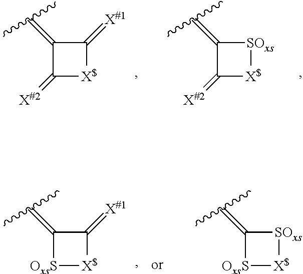

wherein xs is independently selected from 1 (sulfoxide) or 2 (sulfone);
X^{#1} and X^{#2} are independently selected from oxo, thioxo, imino, and CH₂, provided that both X^{#1} and X^{#2} are not each CH₂;
X^{$} is -ethylene-, -propylene-, -ethenyl-, O-methylene, —NR^{#3}-methylene-, O-ethylene, —NR^{#3}-ethylene-, —O-ethylene-O—, —NR^{#3}-ethylene-O—, —NR^{#3}-ethylene-NR^{#3}—, —O—C(═O)—O—, —NR^{#3}—C(═O)—O—, —NR^{#3}—C(═O)—NR^{#3}—, —O—C(═S)—O—, —NR^{#3}—C(═S)—O—, —NR^{#3}—C(═S)—NR^{#3}—, —O-propylene-, —NR^{#3}-propylene-, —O-propylene-O—, —NR^{#3}-propylene-O—, —NR^{#3}-propylene-NR^{#3}—, aryl, cycloalkyl, heteroaryl, or heterocyclyl,
wherein R^{#3} is in each case independently selected from H and C₁₋₄alkyl.
wherein said methylene, ethylene, ethenyl, propylene, propenyl, aryl, cycloalkyl, heteroaryl, and heterocyclyl may be substituted one or more times by F, Cl, Br, I, CN, NH₂, COOH, CONH₂, —(CH₂CH₂O)ₒCH₃ (o=1-16, 1-12, 1-8, 1-6, 1-4, or 1-2), —(CH₂CH₂O)ₒH (o=1-16, 1-12, 1-8, 1-6, 1-4, or 1-2), C₁₋₃alkyl, C₁₋₃haloalkyl, C₁₋₃alkoxy, C₁₋₃haloalkoxy, C₁₋₃aminoalkyl, or C₁₋₃aminohaloalkyl. The skilled person understands that such compounds may be depicted:

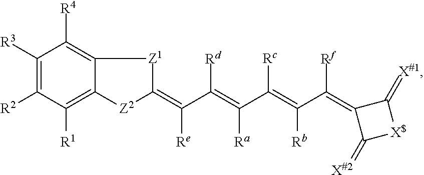

When X¹ and X² together form a ring, exemplary ring systems include:

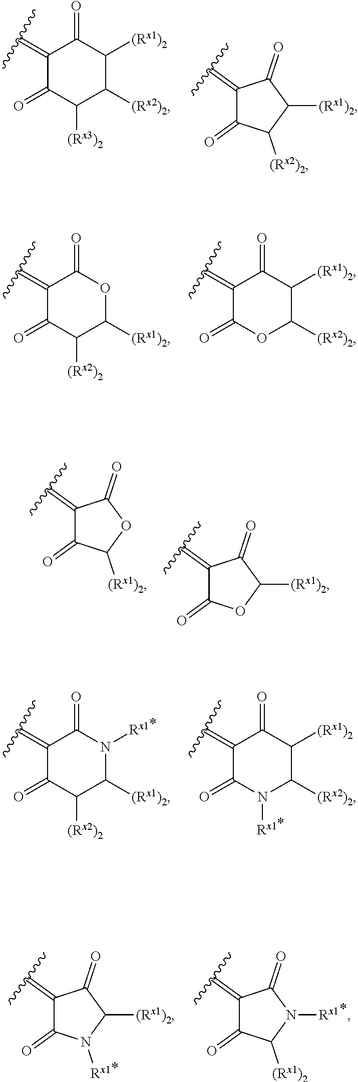

wherein
R^{x1} is in each case independently selected from H, F, Cl, Br, I, CN, NH₂, COOH, CONH₂, —(CH₂CH₂O)ₒCH₃ (o=1-16, 1-12, 1-8, 1-6, 1-4, or 1-2), —(CH₂CH₂O)ₒH (o=1-16, 1-12, 1-8, 1-6, 1-4, or 1-2), C₁₋₃alkyl, C₁₋₃haloalkyl, C₁₋₃alkoxy, C₁₋₃haloalkoxy, C₁₋₃aminoalkyl, or C₁₋₃aminohaloalkyl.
R^{x1}* is in each case independently selected from H, CONH₂, —(CH₂CH₂O)ₒCH₃ (o=1-16, 1-12, 1-8, 1-6, 1-4, or 1-2), —(CH₂CH₂O)ₒH (o=1-16, 1-12, 1-8, 1-6, 1-4, or 1-2), C₁₋₃alkyl, or C₁₋₃haloalkyl,
wherein two geminal R^{x1} groups can together form an oxo, wherein two geminal or adjacent R^{x1} groups can together form a ring, and wherein two adjacent R^{x1} groups can together form a double bond.

In certain embodiments, X¹ and X² together form a ring having the formula:

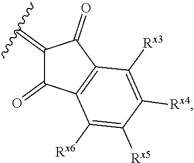

wherein R^{x3} is selected from H, F, Cl, Br, I, CN, NH₂, COOH, CONH₂, —(CH₂CH₂O)ₒCH₃ (o=1-16, 1-12, 1-8, 1-6, 1-4, or 1-2), —(CH₂CH₂O)ₒH (o=1-16, 1-12, 1-8, 1-6, 1-4, or 1-2), C₁₋₈alkyl, C₁₋₃haloalkyl, C₁₋₃alkoxy, C₁₋₃haloalkoxy, C₁₋₃aminoalkyl, or C₁₋₃aminohaloalkyl.
wherein R^{x4} is selected from H, F, Cl, Br, I, CN, NH₂, COOH, CONH₂, —(CH₂CH₂O)ₒCH₃ (o=1-16, 1-12, 1-8, 1-6, 1-4, or 1-2), —(CH₂CH₂O)ₒH (o=1-16, 1-12, 1-8, 1-6, 1-4, or 1-2), C₁₋₃alkyl, C₁₋₃haloalkyl, C₁₋₃alkoxy, C₁₋₃haloalkoxy, C₁₋₃aminoalkyl, or C₁₋₃aminohaloalkyl.
wherein R^{x5} is selected from H, F, Cl, Br, I, CN, NH₂, COOH, CONH₂, —(CH₂CH₂O)ₒCH₃ (o=1-16, 1-12, 1-8, 1-6, 1-4, or 1-2), —(CH₂CH₂O)ₒH (o=1-16, 1-12, 1-8, 1-6, 1-4, or 1-2), C₁₋₃alkyl, C₁₋₃haloalkyl, C₁₋₃alkoxy, C₁₋₃haloalkoxy, C₁₋₃aminoalkyl, or C₁₋₃aminohaloalkyl.
wherein R^{x6} is selected from H, F, Cl, Br, I, CN, NH₂, COOH, CONH₂, —(CH₂CH₂O)ₒCH₃ (o=1-16, 1-12, 1-8, 1-6, 1-4, or 1-2), —(CH₂CH₂O)ₒH (o=1-16, 1-12, 1-8, 1-6, 1-4, or 1-2), C₁₋₃alkyl, C₁₋₃haloalkyl, C₁₋₃alkoxy, C₁₋₃haloalkoxy, C₁₋₃aminoalkyl, or C₁₋₃aminohaloalkyl.
wherein any two or more of R^{x3}, R^{x4}, R^{x5}, and R^{x6} can together form a ring.

In certain embodiments, X¹ and X² together form a ring having the formula:

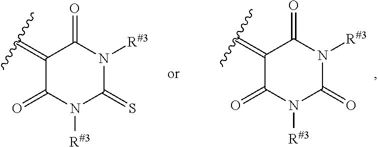

wherein R^{#3} is in each case independently selected from H and C₁₋₄alkyl. In some embodiments, R^{#3} C₁₋₄alkyl substituted one or more times with OH, aryl, F, Cl, Br, I, SO₃H, COOH, NH₂, NH(CH₃), N(CH₃)₂, N+(CH₃)₃, or PO₃H₂.

In certain embodiments, the fluorophore is a compound of Formula (1a):

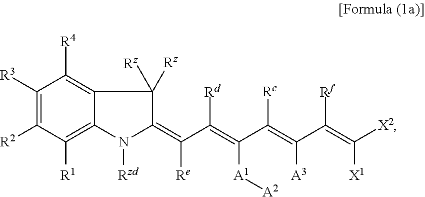

In some embodiments the compound is a compound of Formula (1a1):

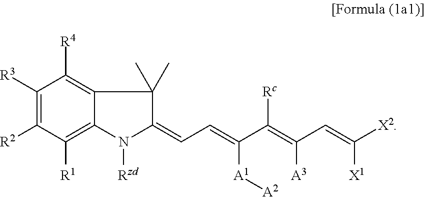

wherein A¹, A², and A³ have the meanings given above. In further embodiments, the fluorophore is a compound of Formula (1a2):

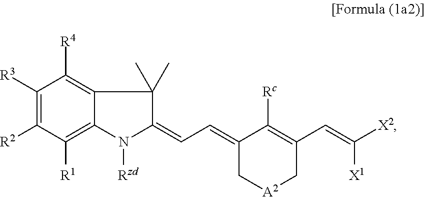

wherein A² is selected from null, O, S, CHR^{a2}, NR^{a2}**, or N⁺(R^{a2}*)₂,
R^{a2} is selected from F, Cl, Br, I, NO₂, CN, R^{a2$}, SR^{a2$}, SeR^{a2$}, OR^{a2$}, N(R^{a2$})₂, SO₂R^{a2$}, SO₂N(R^{a2$})₂, C(O)R^{a2$}; C(O)OR^{a2$}, OC(O)R^{a2$}; C(O)N(R^{a2$})₂, N(R^{a2$})C(O)R^{a2$}, OC(O)N(R^{a2$})₂, N(R^{a2$})C(O)N(R^{a2$})₂, wherein R^{a2$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
wherein both of R^{a2}* is selected from CH₃, or both of R^{a2}* together a pyrrolidine or piperidine ring.

In some embodiments, the fluorophore is a compound of Formula (1a3) or Formula (1a4):

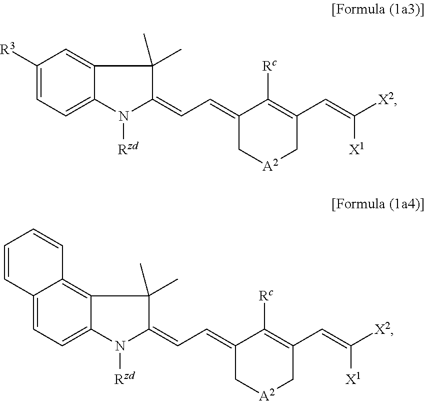

wherein R³ is as defined above.

In certain embodiments for the compounds of Formula (1a), Formula (1a1), Formula (1a2), Formula (1a3), R³ is H, F, Cl, Br, I, C₁₋₃alkyl, C₁₋₃haloalkyl, C₁₋₃alkoxy, or C₁₋₃aminoalkyl.

In certain embodiments for the compounds of Formula (1a), Formula (1a1), Formula (1a2), Formula (1a3), and Formula (1a4) A² is NCH₂CH₂OH, NCH₃, NH, NOH, NOCH₃, NCH₂CH₃, NCH(CH₃)₂, CH, CHC(CH₃)₃, CHCH(CH₃)₂, CHCH₂CH₃, CHCH₂OH, CHOH, CHCH₂CH₂OH, CHOC(CH₃)₃, CHOCH(CH₃)₂, CHOCH₂CH₃, CHCH₂OH, CHOH, CHOCH₂CH₂OH, CHSC(CH₃)₃, CHSCH(CH₃)₂, CHSCH₂CH₃, CHSCH₂CH₂OH, CHSeC(CH₃)₃, CHSeCH(CH₃)₂, CHSeCH₂CH₃, or CHSeCH₂CH₂OH.

In some embodiments of the compound of Formula (1a), Formula (1a1), Formula (1a2), Formula (1a3), and Formula (1a4), A² is null.

In some embodiments of the compound of Formula (1a), Formula (1a1), Formula (1a2), Formula (1a3), and Formula (1a4), X² is COOR^{x2}, wherein R^{x2} is H, C₁₋₃alkyl, C₁₋₃haloalkyl; and X¹ is F, Cl, CN, or C₁₋₃haloalkyl.

In some embodiments of the compound of Formula (1a), Formula (1a1), Formula (1a2), Formula (1a3), and Formula (1a4), R^{zd} is benzyl, C₁₋₈alkyl, preferably C₁₋₄alkyl. In certain embodiments, said C₁₋₈alkyl and C₁₋₄alkyl are n-alkyl groups, e.g., n-propyl, n-butyl, n-pentyl, n-hexyl, n-heptyl, and n-octyl In some embodiments, said benzyl, C₁₋₈alkyl, preferably C₁₋₄alkyl groups are not substituted, while in other embodiments said benzyl, C₁₋₈alkyl, preferably C₁₋₄alkyl groups are substituted one or more times by OH, SO₃H, PO₃H2, COOH, N(CH₃)₂, NH₂, NHCH₃, or a combination thereof.

In some embodiments, the fluorophore is a compound of Formula (1a5) or Formula (1a6):

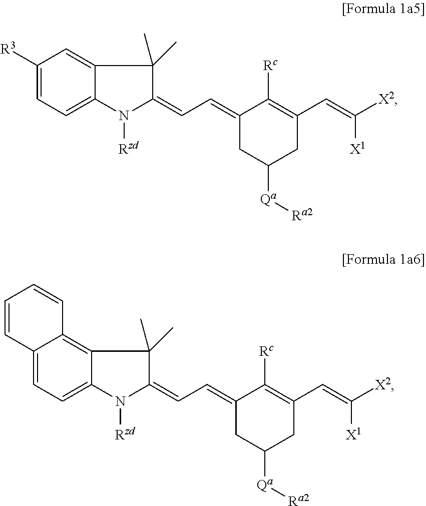

wherein R³, R^{zd}, Q^{a}, X¹, and X² are as defined above. In certain embodiments, R^{a2} is H, F, Cl, Br, I, OH, SO₃H, PO₃H2, COOH, N(CH₃)₂, NH₂, NHCH₃, benzyl, phenyl, or C₁₋₈n-alkyl, preferably C₁₋₄n-alkyl groups. In certain embodiments, said benzyl, phenyl, or C₁₋₈n-alkyl, preferably C₁₋₄n-alkyl groups are not substituted, while in other embodiments said benzyl, phenyl, or C₁₋₈n-alkyl, preferably C₁₋₄n-alkyl groups are substituted one or more times by OH, SO₃H, PO₃H2, COOH, N(CH₃)₂, NH₂, NHCH₃, or a combination thereof.

In some others, the fluorophore is a compound of Formula (1a7) or Formula (1a8):

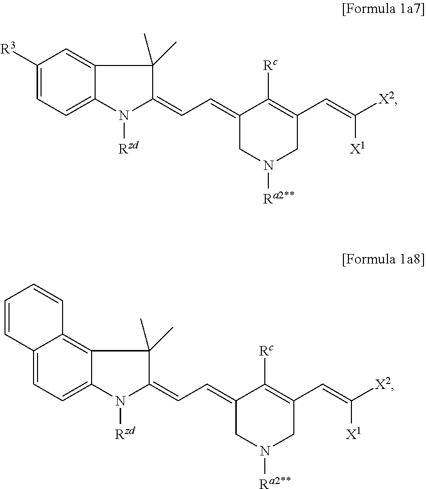

wherein R³, R^{zd}, X¹, and X² are as defined above. In certain embodiments, R^{a2}** is H, benzyl, phenyl, or C₁₋₈alkyl, preferably C₁₋₄alkyl groups. In certain embodiments, said benzyl, phenyl, and C₁₋₈alkyl, preferably C₁₋₄alkyl groups are not substituted, while in other embodiments said benzyl, phenyl, or C₁₋₈alkyl, preferably C₁₋₄alkyl groups are substituted one or more times by OH, SO₃H, PO₃H2, COOH, N(CH₃)₂, NH₂, NHCH₃, or a combination thereof.

In some others, the fluorophore is a compound of Formula (1a9) or Formula (1a10):

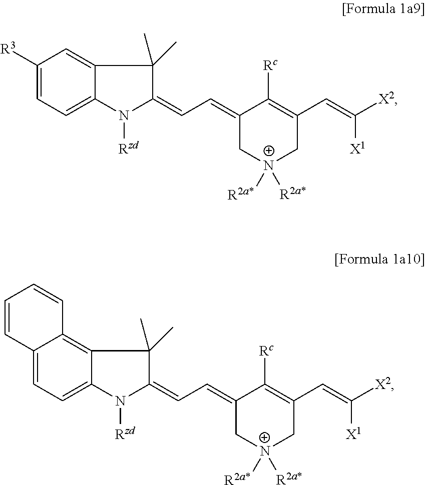

wherein R³, R^{zd}, X¹, and X² are as defined above. In certain embodiments, each R^{a2}* is benzyl, or C₁₋₈alkyl, preferably C₁₋₄alkyl groups, most preferably methyl. In certain embodiments, said benzyl, phenyl, and C₁₋₈alkyl, preferably C₁₋₄alkyl groups are not substituted, while in other embodiments said benzyl, phenyl, or C₁₋₈alkyl, preferably C₁₋₄alkyl, most preferably methyl groups are substituted one or more times by OH, SO₃H, PO₃H2, COOH, N(CH₃)₂, NH₂, NHCH₃, or a combination thereof.

In some embodiments of the compound of Formula (1a), Formula (1a1), Formula (1a2), Formula (1a3), Formula (1a4), of Formula (1a5), Formula (1a6), Formula (1a7), Formula (1a8), Formula (1a9), and Formula (1a10), R^{c} may be F, Cl, or Br. In other embodiments, R^{c} is a OR^{c1}, N(R^{c1})₂, SR^{c1}, wherein R^{c1} is in each case independently selected from hydrogen, C₁₋₈alkyl, aryl, C₁₋₈heteroaryl, C₃₋₈cycloalkyl, or C₁₋₈heterocyclyl.

In some embodiments of the compound of Formula (1a), Formula (1a1), Formula (1a2), Formula (1a3), Formula (1a4), of Formula (1a5), Formula (1a6), Formula (1a7), Formula (1a8), Formula (1a9), and Formula (1a10), R^{c} is OR^{c1}, wherein R^{c1} is selected from H, CH₃, CH₂CH₃, CH(CH₃)₂, CH₂CH₂OH, CH₂COOH, CH₂CH₂COOH.

In some embodiments of the compound of Formula (1a), Formula (1a1), Formula (1a2), Formula (1a3), Formula (1a4), of Formula (1a5), Formula (1a6), Formula (1a7), Formula (1a8), Formula (1a9), and Formula (1a10), R^{c} is NR^{c1}R^{c2} wherein R^{c1} and R^{c2} are different or the same, and are selected from H, CH₃, CH₂CH₃, CH(CH₃)₂, CH₂CH₂OH, CH₂COOH, CH₂CH₂COOH. In certain preferred embodiments, R^{c1} and R^{c2} are the same, and are selected from H, CH₃, CH₂CH₃, CH(CH₃)₂, CH₂CH₂OH, CH₂COOH, CH₂CH₂COOH.

In certain embodiments of the compound of Formula (1a), Formula (1a1), Formula (1a2), Formula (1a3), Formula (1a4), of Formula (1a5), Formula (1a6), Formula (1a7), Formula (1a8), Formula (1a9), and Formula (1a10) R^{c} is NR^{c1}R^{c2}, wherein R^{c2} is hydrogen, and R^{c1} is a C₁₋₈alkyl group substituted one or more times by OH, NH₂, COOH, or a combination thereof. By way of example, R^{c1} can be CH₂CH₂OH, CH(COOH)CH₂OH, CH₂CH(OH)COOH, CH₂CH₂CH(NH₂)COOH, CH₂CH₂CH₂CH₂CH(NH₂)COOH, or CH₂CH₂CH₂CH₂CH₂COOH.

In other embodiments of the compound of Formula (1a), Formula (1a1), Formula (1a2), Formula (1a3), Formula (1a4), of Formula (1a5), Formula (1a6), Formula (1a7), Formula (1a8), Formula (1a9), and Formula (1a10), R^{c} is NR^{c1}R^{c2} and R^{c1} and R^{c2} can together form a heterocyclic ring, for instance a piperidine, pyrrolidine, morpholine, piperazine, which may be further substituted one or more times by F, Cl, Br, I, NO₂, CN, R^{c3}, OR^{c3}, N(R^{c3})₂, SO₂R^{c3}, SO₂N(R^{c3})₂, C(O)R^{c3}; C(O)OR^{c3}, OC(O)R^{c3}; C(O)N(R^{c3})₂, N(R^{c3})C(O)R^{c3}, OC(O)N(R^{c3})₂, N(R^{c3})C(O)N(R^{c3})₂, wherein R^{c3} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

In some embodiments of the compound of Formula (1a), Formula (1a1), Formula (1a2), Formula (1a3), Formula (1a4), of Formula (1a5), Formula (1a6), Formula (1a7), Formula (1a8), Formula (1a9), and Formula (1a10), R^{c} has the formula:

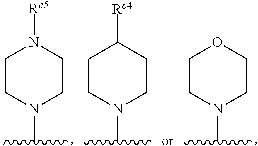

R^{c4} is R^{c4$}, Q⁴-OR^{c4$}, or Q⁴-N(R^{c4$})₂, wherein Q⁴ is null or a C₁₋₄alkylene group, and R^{c4$} is in each case independently selected from hydrogen, C₁₋₈alkyl, aryl, C₁₋₈heteroaryl, C₃₋₈cycloalkyl, or C₁₋₈heterocyclyl; and
C^{c5} is R^{c5$}, wherein Q⁵ is null or a C₁₋₄alkylene group, and R^{c5$} is in each case independently selected from hydrogen, C₁₋₈alkyl, aryl, C₁₋₈heteroaryl, C₃₋₈cycloalkyl, or C₁₋₈heterocyclyl.

In certain preferred embodiments, Q⁴ is null, —CH₂—, —CH₂CH₂— or —CH₂CH₂CH₂—, and R^{c4$} and R^{c5$} are H, OH, NH₂, NHCH₃, N(CH₃)₂, SH, COOH, CH₃, CH₂CH₃, CH(CH₃)₂, C(CH₃)₃, CH₂CH₂OH, CH₂COOH, CH₂CH₂COOH.

In some embodiments, the fluorophore has the formula:

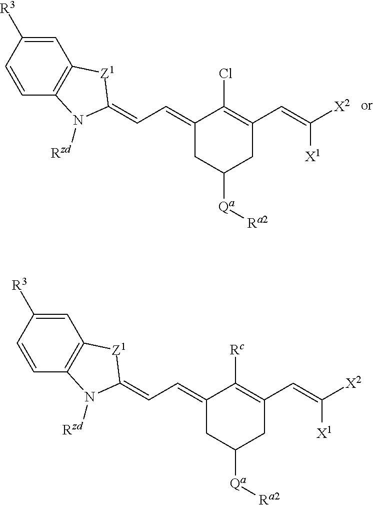

wherein X¹, X², Z¹, Q^{a}, R^{c}, R³, R^{a2}, and R^{zd} are as defined above. For example, the fluorophore may have the formula:

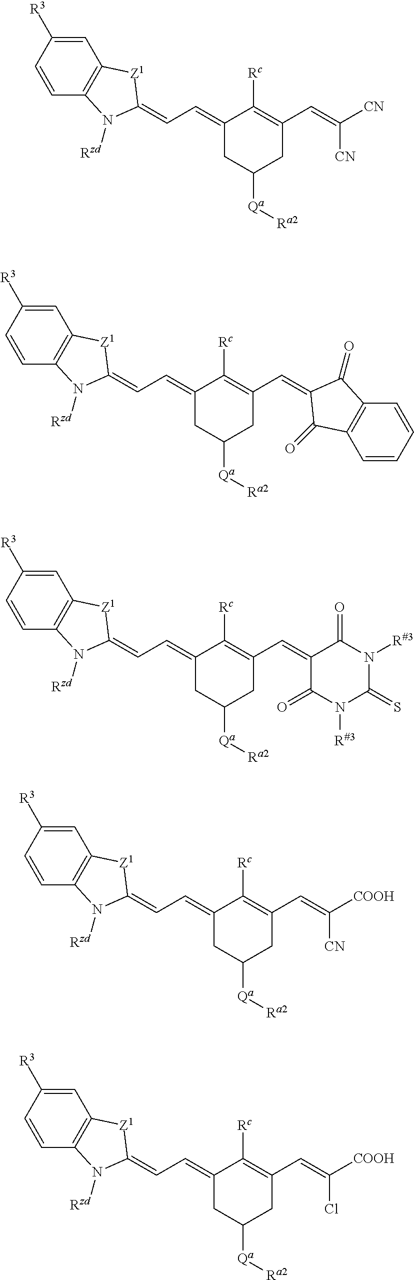

In preferred embodiments, R^{a2} is H, phenyl, pyridinyl, tert-butyl, isopropyl, or benzyl, R³ is H, F, Cl, Br, I, COOH, X¹ is CN, Cl, F, or CF₃, X² is COOH, Z¹ is S, O, —CH═CH—, or C(CH₃)₂, and R^{zd} is unsubstituted C₁₋₈alkyl or C₁₋₈alkyl substituted one or more times by OH, NH₂, COOH, SO₃H, PO₃H₂, or phenyl.

The disclosed compounds can have cytotoxic properties and as such may be effectively used for the treatment of proliferative disorders, including cancer and similar diseases. Exemplary cancers include acute lymphoblastic leukemia (ALL), acute myeloid leukemia (AML), cancer in adrenocortical carcinoma, adrenal cortex cancer, AIDS-related cancers, Kaposi sarcoma, AIDS-related lymphoma, primary CNS lymphoma, anal cancer, appendix cancer, carcinoid tumors, astrocytomas, atypical teratoid/rhabdoid tumor, basal cell carcinoma, skin cancer (nonmelanoma), bile duct cancer, extrahepatic bladder cancer, bladder cancer, bone cancer (includes Ewing sarcoma and osteosarcoma and malignant fibrous histiocytoma), brain tumors, breast cancer, bronchial tumors, Burkitt lymphoma (non-Hodgkin), carcinoid tumor, cardiac (heart) tumors, atypical teratoid/rhabdoid tumor, embryonal tumors, germ cell tumors, lymphoma, primary—cervical cancer, cholangiocarcinoma, chordoma, chronic lymphocytic leukemia (CLL), chronic myelogenous leukemia (CML), chronic myeloproliferative neoplasms, colorectal cancer, colorectal cancer, craniopharyngioma, cutaneous T-cell lymphoma, ductal carcinoma in situ (DCIS), embryonal tumors, central nervous system, endometrial cancer, ependymoma, esophageal, esthesioneuroblastoma, extracranial germ cell tumor, extragonadal germ cell tumor, eye cancer, intraocular melanoma, retinoblastoma, fallopian tube cancer, fibrous histiocytoma of bone, malignant, and osteosarcoma, gallbladder cancer, gastric (stomach) cancer, gastric (stomach) cancer, gastrointestinal carcinoid tumor, gastrointestinal stromal tumors (GIST), gastrointestinal stromal tumors (GIST), germ cell tumors, central nervous system, extracranial, extragonadal, ovarian testicular, gestational trophoblastic disease, gliomas, hairy cell leukemia, head and neck cancer, heart tumors, hepatocellular (liver) cancer, histiocytosis, Langerhans Cell, Hodgkin's lymphoma, hypopharyngeal cancer, intraocular melanoma, islet cell tumors, pancreatic neuroendocrine tumors, Kaposi sarcoma, kidney—langerhans cell histiocytosis, laryngeal cancer, laryngeal cancer and papillomatosis, leukemia, lip and oral cavity cancer, liver cancer (primary), lung cancer, lung cancer, lymphoma—macroglobulinemia, Waldenström—Non-Hodgkin lymphoma, male breast cancer, malignant fibrous histiocytoma of bone and osteosarcoma, melanoma, intraocular (eye), Merkel cell carcinoma, mesothelioma, malignant, mesothelioma, metastatic squamous neck cancer with occult primary, midline tract carcinoma involving NUT gene, mouth cancer, multiple endocrine neoplasia syndromes, multiple myeloma/plasma cell neoplasms, mycosis fungoides, myelodysplastic syndromes, myelodysplastic/myeloproliferative neoplasms and chronic myeloproliferative neoplasms, myelogenous leukemia, chronic (CML), myeloid leukemia, acute (AML), nasal cavity and paranasal sinus cancer, nasopharyngeal cancer, nasopharyngeal cancer, neuroblastoma, non-hodgkin lymphoma, non-small cell lung cancer, oral cancer, lip and oral cavity cancer and oropharyngeal cancer, osteosarcoma and malignant fibrous histiocytoma of bone, ovarian cancer, pancreatic cancer and pancreatic neuroendocrine tumors (islet cell tumors), papillomatosis, paraganglioma, paraganglioma, paranasal sinus and nasal cavity cancer, parathyroid cancer, penile cancer, pharyngeal cancer, pheochromocytoma, pheochromocytoma, pituitary tumor, plasma cell neoplasm/multiple myeloma, pleuropulmonary blastoma, pregnancy and breast cancer, primary central nervous system (CNS) lymphoma, primary peritoneal cancer, prostate cancer, rectal cancer, renal cell (kidney) cancer, retinoblastoma, rhabdomyosarcoma, salivary gland cancer, salivary gland tumors, Ewing sarcoma, Kaposi sarcoma, osteosarcoma, rhabdomyosarcoma, uterine sarcoma, vascular tumors, Sezary syndrome, skin cancer, small cell lung cancer, small intestine cancer, soft tissue sarcoma, squamous cell carcinoma, squamous neck cancer with occult primary, metastatic, stomach (gastric) cancer, stomach (gastric) cancer, T-cell lymphoma, cutaneous, testicular cancer, throat cancer, thymoma and thymic carcinoma, thyroid cancer, ureter and renal pelvis, transitional cell cancer, urethral cancer, uterine cancer, endometrial and uterine sarcoma, vaginal cancer, vaginal cancer, vascular tumors, vulvar cancer, Waldenstrom Macroglobulinemia, Wilms Tumor.

In the field of medical oncology it is normal practice to use a combination of different forms of treatment to treat each patient with cancer. In medical oncology the other component(s) of such conjoint treatment in addition to compositions of the present invention may be, for example, surgery, radiotherapy, chemotherapy, signal transduction inhibitors and/or monoclonal antibodies.

Accordingly, the disclosed compounds may be administered in combination with one or more anticancer agents for example mitotic inhibitors, alkylating agents, anti-metabolites, antisense DNA or RNA, intercalating antibiotics, growth factor inhibitors, signal transduction inhibitors, cell cycle inhibitors, enzyme inhibitors, retinoid receptor modulators, proteasome inhibitors, topoisomerase inhibitors, biological response modifiers, anti-hormones, angiogenesis inhibitors, cytostatic agents anti-androgens, targeted antibodies, HMG-CoA reductase inhibitors, and prenyl-protein transferase inhibitors.

Exemplary anti-cancer agents include nucleoside analogues, antifolates, antimetabolites, topoisomerase I inhibitor, anthracyclines, podophyllotoxins, taxanes, vinca alkaloids, alkylating agents, platinum compounds, proteasome inhibitors, nitrogen mustards & oestrogen analogue, monoclonal antibodies, tyrosine kinase inhibitors, mTOR inhibitors, retinoids, immunomodulatory agents, histone deacetylase inhibitors, and combinations thereof.

In certain embodiments, the anti-cancer agent is selected from one or more of abiraterone acetate, methotrexate, paclitaxel albumin-stabilized nanoparticle, brentuximab vedotin, ado-trastuzumab emtansine, doxorubicin hydrochloride, afatinib dimaleate, everolimus, netupitant, palonosetron hydrochloride, imiquimod, aldesleukin, alectinib, alemtuzumab, melphalan hydrochloride, melphalan, pemetrexed disodium, chlorambucil, aminolevulinic acid, anastrozole, aprepitant, pamidronate disodium, exemestane, nelarabine, arsenic trioxide, ofatumumab, asparaginase Erwinia chrysanthemi, atezolizumab, bevacizumab, axitinib, azacitidine, carmustine, belinostat, bendamustine hydrochloride, bevacizumab, bexarotene, tositumomab, bicalutamide, bleomycin, blinatumomab, blinatumomab, bortezomib, bosutinib, busulfan, cabazitaxel, cabozantinib, alemtuzumab, irinotecan hydrochloride, capecitabine, fluorouracil, carboplatin, carfilzomib, bicalutamide, lomustine, ceritinib, daunorubicin hydrochloride, cetuximab, chlorambucil, cyclophosphamide, clofarabine, cobimetinib, dactinomycin, cobimetinib, crizotinib, ifosfamide, ramucirumab, cytarabine, dabrafenib, dacarbazine, decitabine, daratumumab, dasatinib, daunorubicin hydrochloride, decitabine, efibrotide sodium, defibrotide sodium, degarelix, denileukin diftitox, denosumab, dexamethasone, dexrazoxane hydrochloride, dinutuximab, docetaxel, doxorubicin hydrochloride, dacarbazine, rasburicase, epirubicin hydrochloride, elotuzumab, oxaliplatin, eltrombopag olamine, aprepitant, elotuzumab, enzalutamide, epirubicin hydrochloride, cetuximab, eribulin mesylate, vismodegib, erlotinib hydrochloride, etoposide, raloxifene hydrochloride, melphalan hydrochloride, toremifene, panobinostat, fulvestrant, letrozole, filgrastim, fludarabine phosphate, flutamide, methotrexate, pralatrexate, recombinant hpv quadrivalent vaccine, recombinant hpv nonavalent vaccine, obinutuzumab, gefitinib, gemcitabine hydrochloride, gemtuzumab ozogamicin, afatinib dimaleate, imatinib mesylate, glucarpidase, goserelin acetate, eribulin mesylate, trastuzumab, topotecan hydrochloride, palbociclib, ibritumomab tiuxetan, ibrutinib, ponatinib hydrochloride, idarubicin hydrochloride, idelalisib, imiquimod, axitinib, recombinant interferon alfa-2b, tositumomab, ipilimumab, gefitinib, romidepsin, ixabepilone, ixazomib citrate, ruxolitinib phosphate, cabazitaxel, ado-trastuzumab emtansine, palifermin, pembrolizumab, lanreotide acetate, lapatinib ditosylate, lenalidomide lenvatinib mesylate, leuprolide acetate, olaparib, vincristine sulfate, procarbazine hydrochloride, mechlorethamine hydrochloride, megestrol acetate, trametinib, mercaptopurine, temozolomide, mitoxantrone hydrochloride, plerixafor, busulfan, azacitidine, gemtuzumab ozogamicin, vinorelbine tartrate, necitumumab, nelarabine, sorafenib tosylate, nilotinib, ixazomib citrate, nivolumab, romiplostim, obinutuzumab, ofatumumab, olaparib, omacetaxine mepesuccinate, pegaspargase, ondansetron hydrochloride, osimertinib, panitumumab, panobinostat, peginterferon alfa-2b, pembrolizumab, pertuzumab, plerixafor, pomalidomide, ponatinib hydrochloride, necitumumab, pralatrexate, procarbazine hydrochloride, aldesleukin, denosumab, ramucirumab, rasburicase, regorafenib, lenalidomide, rituximab, rolapitant hydrochloride, romidepsin, ruxolitinib phosphate, siltuximab, dasatinib, sunitinib malate, thalidomide, dabrafenib, osimertinib, talimogene, atezolizumab, temsirolimus, thalidomide, dexrazoxane hydrochloride, trabectedin, trametinib, trastuzumab, lapatinib ditosylate, dinutuximab, vandetanib, rolapitant hydrochloride, bortezomib, venetoclax, crizotinib, enzalutamide, ipilimumab, trabectedin, ziv-aflibercept, idelalisib, ceritinib, and pharmaceutically acceptable salts thereof.

The disclosed compounds fluoresce at a wavelength of 800 nm or more, and as such are useful as imaging agents. According, in some aspects the invention relates to methods of diagnosing or imaging a tumor or other condition in a body. Embodiments including one or more basic atoms (e.g., basic nitrogen and the like) can further be used a pH-sensitive probes.

As used herein, terms “treat” or “treatment” refer to therapeutic, prophylactic, palliative or preventative measures. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, the term “mammal” refers to a warm-blooded animal that has or is at risk of developing a disease described herein and includes, but is not limited to, guinea pigs, dogs, cats, rats, mice, hamsters, and primates, including humans.

Compounds of the invention may be administered by any convenient route, e.g. into the gastrointestinal tract (e.g. rectally or orally), the nose, lungs, musculature or vasculature, or transdermally or dermally. Compounds may be administered in any convenient administrative form, e.g. tablets, powders, capsules, solutions, dispersions, suspensions, syrups, sprays, suppositories, gels, emulsions, patches etc. Such compositions may contain components conventional in pharmaceutical preparations, e.g. diluents, carriers, pH modifiers, sweeteners, bulking agents, and further active agents. If parenteral administration is desired, the compositions will be sterile and in a solution or suspension form suitable for injection or infusion. Such compositions form a further aspect of the invention.

The compounds disclosed herein may be formulated in a wide variety of pharmaceutical compositions for administration to a patient. Such compositions include, but are not limited to, unit dosage forms including tablets, capsules (filled with powders, pellets, beads, mini-tablets, pills, micro-pellets, small tablet units, multiple unit pellet systems (MUPS), disintegrating tablets, dispersible tablets, granules, and microspheres, multiparticulates), sachets (filled with powders, pellets, beads, mini-tablets, pills, micro-pellets, small tablet units, MUPS, disintegrating tablets, dispersible tablets, granules, and microspheres, multiparticulates), powders for reconstitution, transdermal patches and sprinkles, however, other dosage forms such as controlled release formulations, lyophilized formulations, modified release formulations, delayed release formulations, extended release formulations, pulsatile release formulations, dual release formulations and the like. Liquid or semisolid dosage form (liquids, suspensions, solutions, dispersions, ointments, creams, emulsions, microemulsions, sprays, patches, spot-on), injection preparations, parenteral, topical, inhalations, buccal, nasal etc. may also be envisaged under the ambit of the invention.

Suitable excipients may be used for formulating the dosage forms according to the present invention such as, but not limited to, surface stabilizers or surfactants, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, channeling agents, coating agents or combinations thereof.

The compounds disclosed herein may be administered by a number of different routes. For instance, the compounds may be administered orally, topically, transdermally, intravenously, subcutaneously, by inhalation, or by intracerebroventricular delivery.

In some embodiments, the compounds disclosed herein may be formulated as nanoparticles. The nanoparticles may have an average particle size from 1-1,000 nm, preferably 10-500 nm, and even more preferably from 10-200 nm.

The compounds may be administered to a patient systemically, e.g., by oral or intravenous administration, topically, i.e., by application of a cream, lotion or the like, or locally, e.g., by direct perfusion of a composition containing the compound to a target tissue.

The present invention further provides a pharmaceutical composition, which comprises a compound of Formula I or a pharmaceutically acceptable salt thereof, as defined hereinabove. In one embodiment, the pharmaceutical composition includes the disclosed compounds together with a pharmaceutically acceptable diluent or carrier.

The present invention further provides a disclosed compound or a pharmaceutically acceptable salt thereof, for use in therapy.

In one embodiment, the invention provides the disclosed compounds or a pharmaceutically acceptable salt thereof, for use in the treatment of cancer in a mammal. In one embodiment, the cancer is neuroblastoma. In one embodiment, the cancer is ovarian cancer. In one embodiment, the cancer is pancreatic cancer. In one embodiment, the cancer is colorectal cancer. In one embodiment, the cancer is prostate cancer.

Another embodiment of the present invention provides the use of the disclosed compounds, or a pharmaceutically acceptable salt thereof, in the manufacture of a medicament for the treatment of cancer in a mammal.

The compounds disclosed herein may be prepared by condensation, for instance under Vilsmeier Haack conditions, of an appropriate acetone derivative with aldehydes (or masked aldehyde equivalents), followed by condensation with X¹—CH₂—X¹:

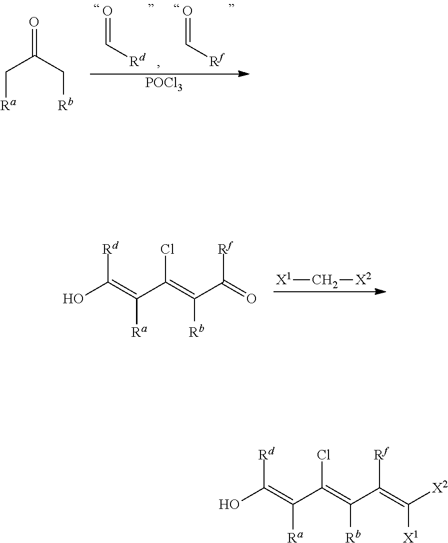

wherein R^{a}, R^{b}, R^{d}, R^{f}, X¹, and X² are as defined above. The resulting product may be condensed with an appropriate heterocycle, and if desired, the meso chlorine atom exchanged with a different R^{c} group:

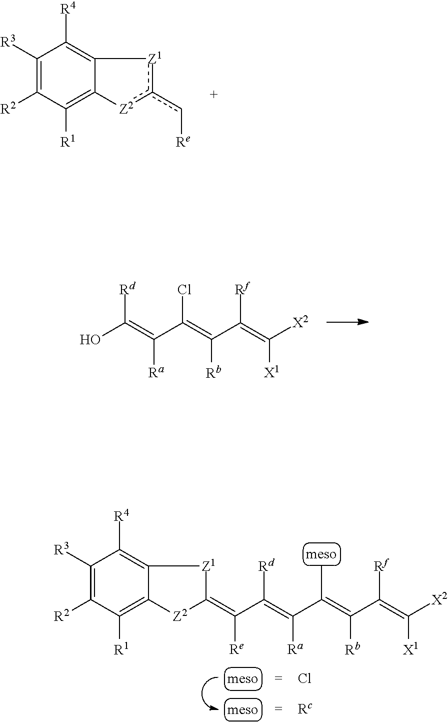

The condensation with the heterocycle may be carried out in mixtures of an amine base and ethanolic solvent, optionally at elevated temperature (reflux).

In some implementations, compounds were prepared according under the following conditions:

### EXAMPLES

The following examples are for the purpose of illustration of the invention only and are not intended to limit the scope of the present invention in any manner whatsoever.

### Example 1: Synthesis of Linker-Acceptor Compounds

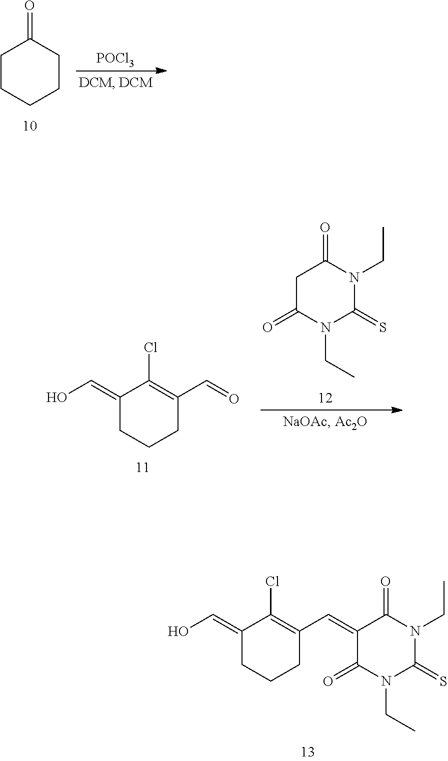

9 mL of DCM and 18 ml of DMF were combined in a 50 mL round bottomed flask in an ice bath. The solution was let to stir in ice bath for 15 minutes. Then 15 mL (58 mmol) of POCl₃ was add slowly into the solution: 1 mL of POCl₃ was added every few minutes to make up to 15 ml of POCl₃ while stirring in reflux, and then stirred for further 30 minutes. After 30 minutes, 4.5 mL (48 mmol) of cyclohexanone 10 was added slowly into Vilsmeier reagent. The flask was taken off the ice bath and stirred under reflux at 80° C. for 4 hours. Upon heating, the reaction mixture turned bright red in color. The reaction was monitored with thin layered chromatography (TLC). With cyclohexanone as the starting material, the reaction mixture was compared on TLC after every hour to see the consumption of cyclohexanone. When the starting material was consumed and yellow product was formed as seen on TLC, the reaction was stopped, and the reaction mixture was poured over iced water in a large Erlenmeyer flask. It was let stir overnight. The overnight stirred bright red mixture in iced water had turned bright yellow. The yellow precipitate 11 was collected by vacuum filtration and was washed with ice cold water.

The linker 11 was then reacted with 1,3-diethyl-2-thiobarbituric acid 12 to form the linker acceptor complex 13. 3 g (17.4 mmol) of linker 11 was taken into a 100 mL round bottomed flask with 3 mL of acetic anhydride. 3.48 g (17.4 mmol) of 12 was added into this mixture and let stir until dissolved. Then 2 equivalents of NaOAc weighing 2.85 g (34.8 mmol) was added into the mixture, and refluxed at 70° C. The reaction progress was monitored with TLC after every hour. Once the TLC showed majority of starting material, compound 11 was consumed and product 13 was formed, the reaction was stopped after 6 hours.² This resulting reaction mixture was poured onto iced water and let stir overnight. Product 13 was dark green in color as solid precipitate. The crude product was purified by crystallization in methanol and confirmed by ¹H NMR.

Using similar protocols, linker-acceptor compounds having a malononitrile, cyanoacetic acid, and indane-1,3-dione functionality were prepared.

### Example 2: Synthesis of Red-Shifted Fluorophores

(E)-3-((E)-2-chloro-3-(hydroxymethylene)cyclohex-1-en-1-yl)-2-cyanoacrylic acid (1 eq) and indolium heterocyclic salt (1.2 eq) were dissolved in ethanol (5 mL) and refluxed at 70° C. with the addition of 3 drops of triethylamine. The progress of the reaction was monitored by using UV-Vis spectrophotometer. The reaction was continued until the peak around 780 nm was observed and the peak around 440 nm due to heterocyclic salt was disappeared. The reaction was completed after 6 h. After the completion of the reaction, the excess ethanol was evaporated by using rotary evaporator. The crude compound was then dissolved in minimal amount of methanol and diethyl ether was added to it to obtain pure green crystals of donor-π-acceptor fluorophores.

(E)-2-((2-chloro-3-(hydroxymethylene) cyclohex-1-en-1-yl)methylene)malononitrile (1 eq) and indolium heterocyclic salt (1.2 eq) were dissolved in ethanol (5 mL) and refluxed at 70° C. with the addition of 3 drops of triethylamine. The progress of the reaction was monitored by using UV-Vis spectrophotometer. The reaction was continued until the peak around 780 nm was observed and the peak around 440 nm due to heterocyclic salt was disappeared. The reaction was completed after 6 h. After the completion of the reaction, the excess ethanol was evaporated by using rotary evaporator. The compound was then dissolved in minimal amount of methanol and diethyl ether was added to it to obtain crude powder of donor-π-acceptor fluorophores. The fluorophores were purified by using column chromatography with solvent system of DCM and acetone.

### Example 3: Meso Functionalization with piperidin-4-yl-methanol

Meso-chloro substituted fluorophores (1 eq) and piperidin-4-yl-methanol (5 eq) were dissolved in DMF (2 mL) and heated at 85° C. under nitrogen atmosphere. The progress of the reaction was monitored by using UV-Vis spectrophotometer and TLC. The reaction was continued until the peak around 660 nm was observed and the starting fluorophore peak around 780 nm was disappeared and in addition, the starting fluorophore spot was disappeared in TLC. The reaction was completed after 3 h. After the completion of the reaction, diethyl ether was added to the reaction mixture and stirred for an hour to get solid form of crude product. The crude product was purified by using column chromatography with solvent system of DCM and acetone to obtain purple powder of pure meso-modified fluorophores.

### Example 3: Meso Functionalization with pyridine-4-thiol

Pyridine-4-thiol (1 eq) and sodium methoxide (2 eq) were dissolved in DMF (1 mL) and stirred at 0° C. under nitrogen atmosphere for 30 min. The two donor-π-acceptor fluorophores were dissolved in DMF (2 mL), added dropwise to the reaction mixture, and stirred at room temperature as shown in Equation 20. The progress of the reaction was monitored by using UV-Vis spectrophotometer and TLC. The reaction was continued until the peak around 800 nm was observed and the starting fluorophore peak around 780 nm was disappeared and in addition, the starting fluorophore spot was disappeared in TLC. The reaction was completed after 2 h. After the completion of the reaction, diethyl ether was added to the reaction mixture and stirred for an hour to get solid form of crude product. The crude product was purified by using column chromatography with solvent system of DCM and acetone to obtain green powder of pure meso-modified fluorophores.

Following these protocols, the following compounds were prepared:

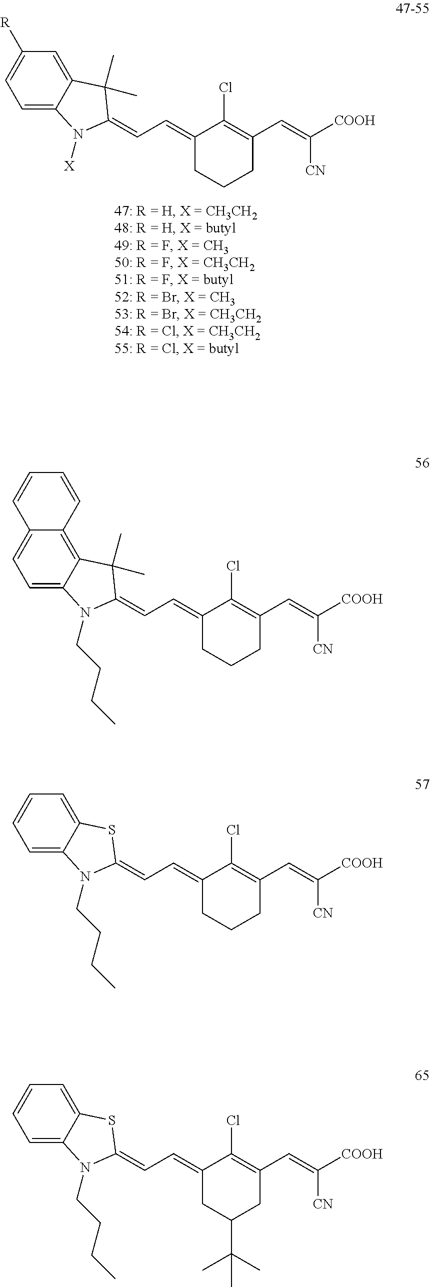

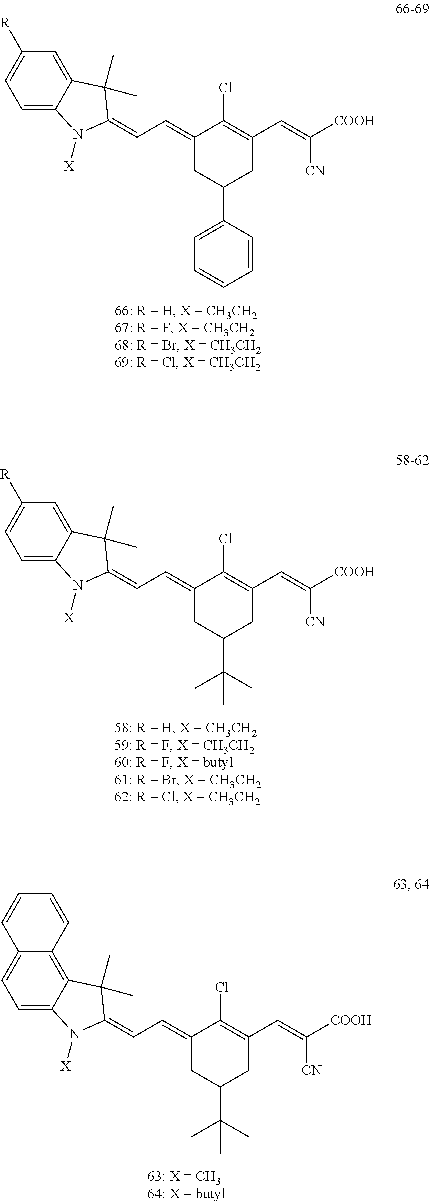

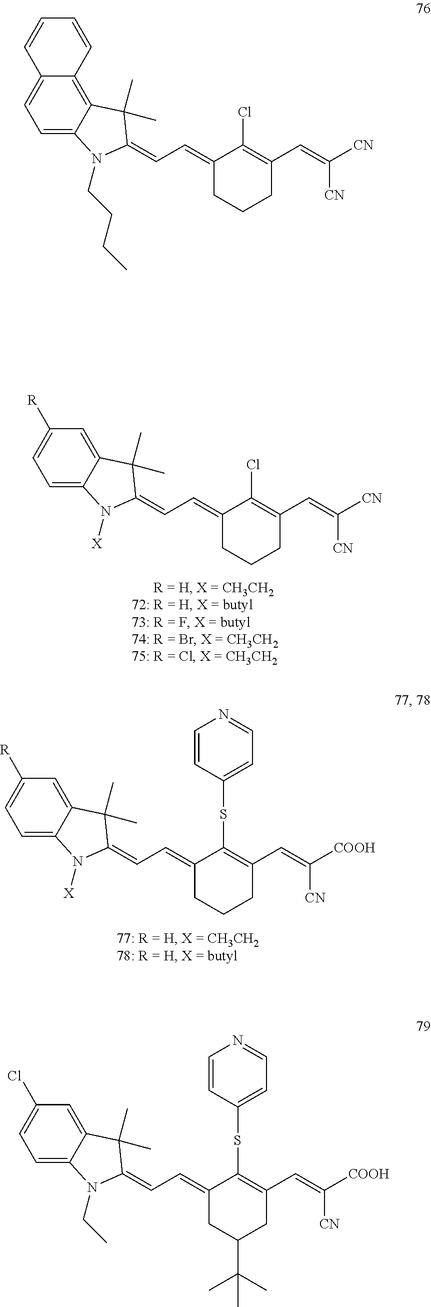

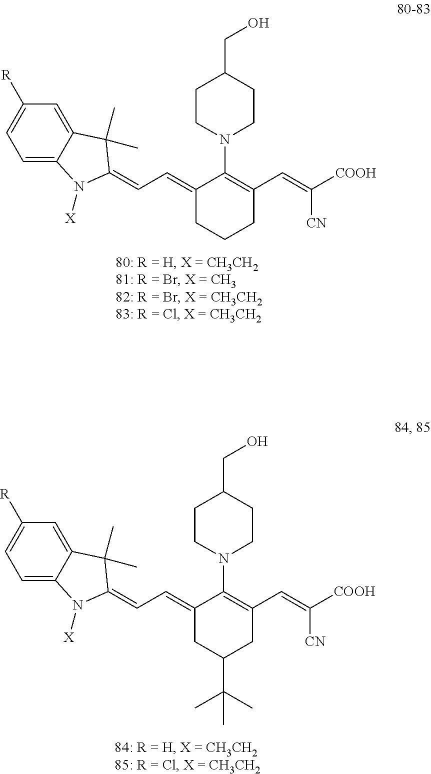

Other exemplary compounds include:

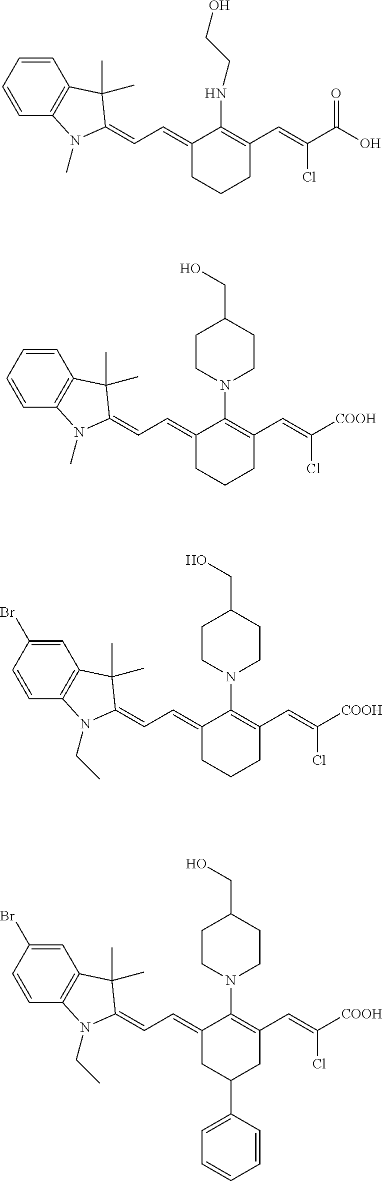 [SMILES: CN1/C(=C/C=C2\CCCC(/C=C(\Cl)C(=O)O)=C2NCCO)C(C)(C)c2ccccc21 ; CN1/C(=C/C=C2\CCCC(/C=C(\Cl)C(=O)O)=C2N2CCC(CO)CC2)C(C)(C)c2ccccc21 ; CCN1/C(=C/C=C2\CCCC(/C=C(\Cl)C(=O)O)=C2N2CCC(CO)CC2)C(C)(C)c2cc(Br)ccc21 ; CCN1/C(=C/C=C2\CC(c3ccccc3)CC(/C=C(\Cl)C(=O)O)=C2N2CCC(CO)CC2)C(C)(C)c2cc(Br)ccc21]

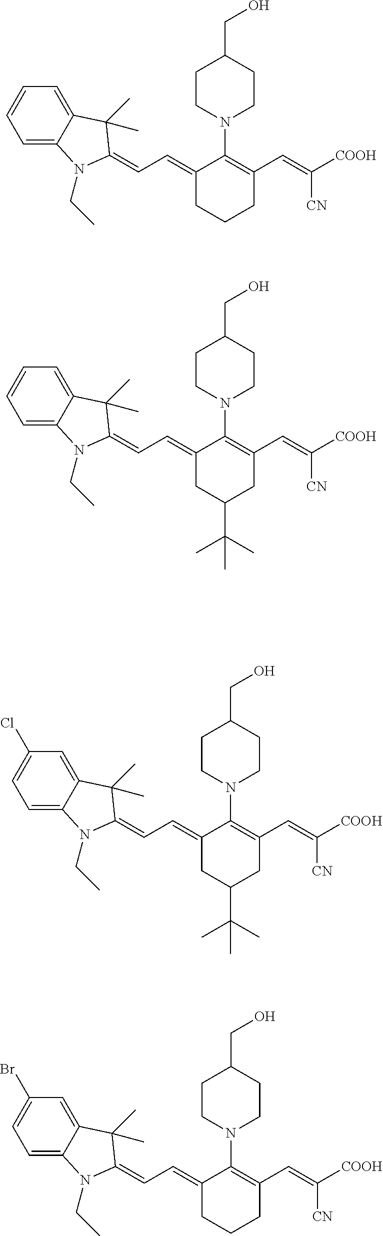 [SMILES: CCN1/C(=C/C=C2\CCCC(/C=C(\C#N)C(=O)O)=C2N2CCC(CO)CC2)C(C)(C)c2ccccc21 ; CCN1/C(=C/C=C2\CC(C(C)(C)C)CC(/C=C(\C#N)C(=O)O)=C2N2CCC(CO)CC2)C(C)(C)c2ccccc21 ; CCN1/C(=C/C=C2\CC(C(C)(C)C)CC(/C=C(\C#N)C(=O)O)=C2N2CCC(CO)CC2)C(C)(C)c2cc(Cl)ccc21 ; CCN1/C(=C/C=C2\CCCC(/C=C(\C#N)C(=O)O)=C2N2CCC(CO)CC2)C(C)(C)c2cc(Br)ccc21]

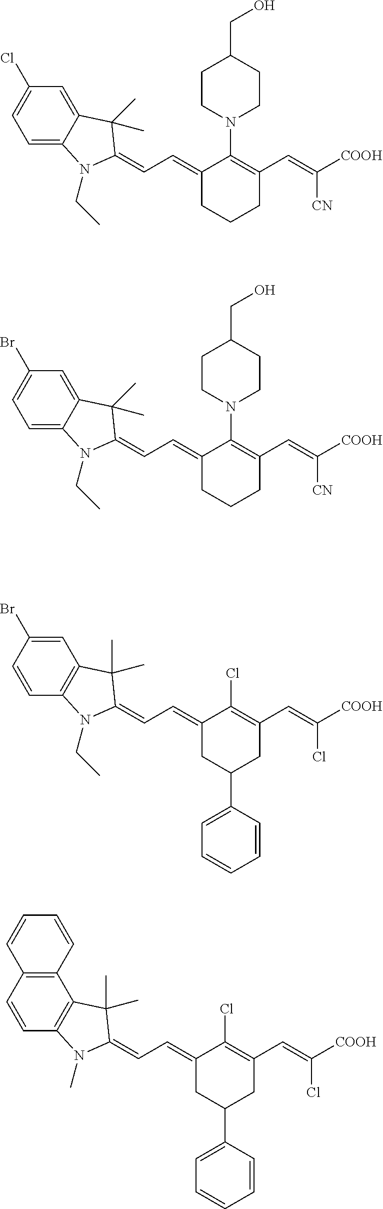 [SMILES: CCN1/C(=C/C=C2\CCCC(/C=C(\C#N)C(=O)O)=C2N2CCC(CO)CC2)C(C)(C)c2cc(Cl)ccc21 ; CCN1/C(=C/C=C2\CCCC(/C=C(\C#N)C(=O)O)=C2N2CCC(CO)CC2)C(C)(C)c2cc(Br)ccc21 ; CCN1/C(=C/C=C2\CC(c3ccccc3)CC(/C=C(\Cl)C(=O)O)=C2Cl)C(C)(C)c2cc(Br)ccc21 ; CN1/C(=C/C=C2\CC(c3ccccc3)CC(/C=C(\Cl)C(=O)O)=C2Cl)C(C)(C)c2c1ccc1ccccc21]

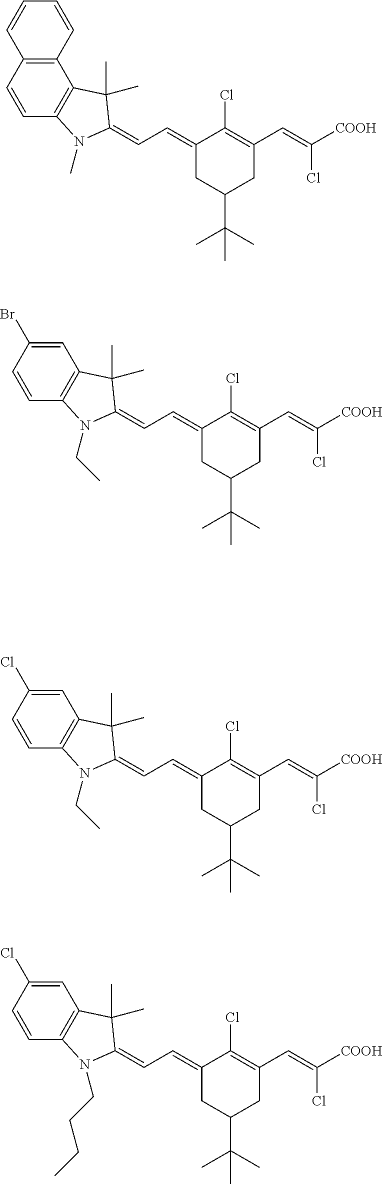 [SMILES: CN1/C(=C/C=C2\CC(C(C)(C)C)CC(/C=C(\Cl)C(=O)O)=C2Cl)C(C)(C)c2c1ccc1ccccc21 ; CCN1/C(=C/C=C2\CC(C(C)(C)C)CC(/C=C(\Cl)C(=O)O)=C2Cl)C(C)(C)c2cc(Br)ccc21 ; CCN1/C(=C/C=C2\CC(C(C)(C)C)CC(/C=C(\Cl)C(=O)O)=C2Cl)C(C)(C)c2cc(Cl)ccc21 ; CCCCN1/C(=C/C=C2\CC(C(C)(C)C)CC(/C=C(\Cl)C(=O)O)=C2Cl)C(C)(C)c2cc(Cl)ccc21]

### Example 4: Evaluation of Red-Shifted Fluorophores

Optical properties of the fluorophores was measured in ethanol, and compared to ICG (indocyanine green).

| Ex | λ_{abs} (nm) | λₑₘ (nm) | Stokes Shift (nm) | ϵ (cm⁻¹ M⁻¹) | Φ^{a} (%) |
|---|---|---|---|---|---|
| 47 | 780 | 800 | 20 | 179,500 | 12.3 |
| 48 | 785 | 802 | 17 | 181,400 | 12.1 |
| 49 | 780 | 795 | 15 | 195,100 | 12.6 |
| 50 | 780 | 799 | 14 | 165,300 | 12.2 |
| 51 | 785 | 802 | 17 | 176,400 | 11.9 |
| 52 | 790 | 806 | 16 | 153,600 | 11.8 |
| 53 | 790 | 807 | 17 | 171,000 | 11.8 |
| 54 | 790 | 805 | 15 | 156,500 | 12.0 |
| 55 | 795 | 808 | 13 | 115,500 | 11.5 |
| 56 | 820 | 827 | 7 | 151,100 | 9.6 |
| 57 | 800 | 814 | 14 | 198,700 | 10.5 |
| 58 | 780 | 797 | 17 | 138,200 | 12.7 |
| 59 | 780 | 799 | 19 | 123,000 | 12.5 |
| 60 | 785 | 803 | 18 | 137,400 | 12.6 |
| 61 | 790 | 805 | 15 | 123,500 | 11.4 |
| 62 | 790 | 805 | 15 | 114,100 | 11.5 |
| 63 | 815 | 821 | 6 | 167,100 | 9.8 |
| 64 | 820 | 828 | 8 | 146,300 | 7.3 |
| 65 | 800 | 812 | 12 | 192,000 | 10.5 |
| 66 | 780 | 795 | 15 | 155,700 | 12.4 |
| 67 | 780 | 796 | 16 | 124,700 | 12.3 |
| 68 | 790 | 805 | 15 | 167,800 | 12.1 |
| 69 | 790 | 801 | 11 | 136,500 | 11.7 |
| 47 | 780 | 800 | 20 | 179,500 | 12.3 |
| 50 | 780 | 799 | 14 | 165,300 | 12.2 |
| 53 | 790 | 807 | 17 | 171,000 | 11.8 |
| 54 | 790 | 805 | 15 | 156,500 | 12.0 |
| 58 | 780 | 797 | 17 | 138,200 | 12.7 |
| 59 | 780 | 799 | 19 | 123,000 | 12.5 |
| 61 | 790 | 805 | 15 | 123,500 | 11.4 |
| 62 | 790 | 805 | 15 | 114,100 | 11.5 |
| 66 | 780 | 795 | 15 | 155,700 | 12.4 |
| 67 | 780 | 796 | 16 | 124,700 | 12.3 |
| 68 | 790 | 805 | 15 | 167,800 | 12.1 |
| 69 | 790 | 801 | 11 | 136,500 | 11.7 |
| 70 | 780 | 798 | 18 | 161,200 | 12.5 |
| 71 | 780 | 797 | 17 | 144,100 | 12.1 |
| 72 | 785 | 800 | 15 | 143,900 | 12.1 |
| 73 | 785 | 802 | 17 | 168,800 | 12.2 |
| 74 | 790 | 809 | 19 | 152,300 | 11.3 |
| 75 | 790 | 805 | 15 | 145,400 | 11.5 |
| 76 | 820 | 826 | 6 | 146,400 | 8.8 |
| 77 | 800 | 815 | 15 | 134,400 | 11.5 |
| 78 | 800 | 815 | 15 | 166,300 | 11.3 |
| 79 | 805 | 818 | 13 | 147,300 | 11.3 |
| 80 | 685 | 786 | 101 | 78,200 | 16.7 |
| 81 | 665 | 791 | 126 | 64,200 | 18.3 |
| 82 | 675 | 794 | 124 | 61,000 | 17.3 |
| 83 | 670 | 792 | 122 | 75,300 | 17.4 |
| 84 | 660 | 784 | 124 | 75,100 | 18.6 |
| 85 | 645 | 788 | 143 | 60,000 | 19.9 |

The compounds disclosed herein exhibit solvatochromism

λ_{abs} (nm)

| Fluorophore | Methanol | Acetonitrile | Ethanol | DMSO |
|---|---|---|---|---|
| 70 | 775 | 775 | 780 | 790 |
| 74 | 785 | 790 | 790 | 800 |
| 76 | 820 | 820 | 820 | 835 |
| 77 | 795 | 790 | 800 | 810 |
| 78 | 800 | 795 | 800 | 810 |
| 79 | 800 | 800 | 805 | 815 |
| 80 | 675 | 660 | 685 | 690 |
| 82 | 665 | 640 | 675 | 670 |
| 84 | 650 | 640 | 660 | 665 |

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1-127. (canceled)

128. A compound having the formula:
[donor]-[linker]-[acceptor],
or a pharmaceutically acceptable salt thereof,
wherein:
[donor] has the formula: 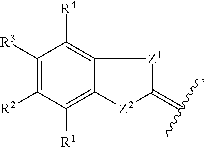
Z² is N—R^{zd} and Z¹ is O, S, C(R^{z})₂ N—R^{z}, N═CR^{z1}, or C(CR)═C(CR^{z1}); or
Z² is null and Z¹ is Z³—N(R^{zd})—, wherein Z³ is N═CR^{z3}*, C(CR^{z3})═C(CR^{z3}); O, S, or C(R^{z})₂;
wherein R^{z} and R^{zd} are each case independently optionally substituted C₁₋₆alkyl;
R^{z1} is in case independently selected from F, Cl, Br, I, NO₂, CN, or R^{z1}*, wherein R^{z1}* is hydrogen or C₁₋₈alkyl;
R^{z3} is in each case independently selected from F, Cl, Br, I, NO₂, CN, or R^{z3}, wherein R^{z3}* is hydrogen or C₁₋₈alkyl;
R¹ is F, Cl, Br, I, NO₂, CN, R^{1a}, OR^{1a}, N(R^{1a})₂, SO₃R^{1a}, SO₂R^{1a}, SO₂N(R^{1a})₂, C(O)R^{1a}; C(O)OR^{1a}, OC(O)R^{1a}; C(O)N(R^{1a})₂, N(R^{1a})C(O)R^{1a}, OC(O)N(R^{1a})₂, N(R^{1a})C(O)N(R^{1a})₂, wherein R^{1a} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
R² is F, Cl, Br, I, NO₂, CN, R^{2a}, OR^{2a}, N(R^{2a})₂, SO₃R^{2a}, SO₂R^{2a}, SO₂N(R^{2a})₂, C(O)R^{2a}; C(O)OR^{2a}, OC(O)R^{2a}; C(O)N(R^{2a})₂, N(R^{2a})C(O)R^{2a}, OC(O)N(R^{2a})₂, N(R^{2a})C(O)N(R^{2a})₂, wherein R^{2a} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
R³ is F, Cl, Br, I, NO₂, CN, R^{3a}, OR^{3a}, N(R^{3a})₂, SO₃R^{3a}, SO₂R^{3a}, SO₂N(R^{3a})₂, C(O)R^{3a}; C(O)OR^{3a}, OC(O)R^{3a}; C(O)N(R^{3a})₂, N(R^{3a})C(O)R^{3a}, OC(O)N(R^{3a})₂, N(R^{3a})C(O)N(R^{3a})₂, wherein R^{3a} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl; and
R⁴ is F, Cl, Br, I, NO₂, CN, R^{4a}, OR^{4a}, N(R^{4a})₂, SO₃R^{4a}, SO₂R^{4a}, SO₂N(R^{4a})₂, C(O)R^{4a}; C(O)OR^{4a}, OC(O)R^{4a}; C(O)N(R^{4a})₂, N(R^{4a})C(O)R^{4a}, OC(O)N(R^{4a})₂, N(R^{4a})C(O)N(R^{4a})₂, wherein R^{4a} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
[acceptor] has the formula: 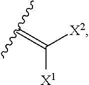
wherein X¹ is Cl, F, Br, I, Q-C(═O)R^{x1}, Q-C(═O)OR^{x1}, Q-SO₂R^{x1}, Q-C(═O)N(R^{x1})₂, Q-C₁₋₃haloalkyl; Q-CN, or Q-NO₂;
X² is Cl, F, Br, I, Q-C(═O)R^{x2}, Q-C(═O)OR^{x2}, Q-SO₂R^{x2}, Q-C(═O)N(R^{x2})₂, Q-C₁₋₃haloalkyl; Q-CN, or Q-NO₂,
wherein Q is in each case independently selected from null, CH═CH, or C≡C;
R^{x1} is in each case independently selected from H, optionally substituted C₁₋₆alkyl, optionally substituted C₁₋₆haloalkyl; optionally substituted C₁₋₁₀aryl; optionally substituted C₃₋₁₀cycloalkyl; optionally substituted C₁₋₁₀heteroaryl; optionally substituted C₁₋₁₀heterocyclyl;
R^{x2} is in each case independently selected from H, optionally substituted C₁₋₆alkyl, optionally substituted C₁₋₆haloalkyl; optionally substituted C₁₋₁₀aryl; optionally substituted C₃₋₁₀cycloalkyl; optionally substituted C₁₋₁₀heteroaryl; optionally substituted C₁₋₁₀heterocyclyl;
[linker] has the formula: 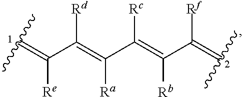
wherein the wavy line marked ‘1’ represents the point of attachment to the [donor] and the wavy line marker ‘2’ represents the point of attachment to the acceptor;
R^{a} is F, Cl, Br, I, NO₂, CN, R^{a1}, OR^{a1}, SR^{a1}, N(R^{a1})₂, SO₂R^{a1}, SO₂N(R^{a1})₂, C(O)R^{a1}; C(O)OR^{a1}, OC(O)R^{a1}; C(O)N(R^{a1})₂, N(R^{a1})C(O)R^{a1}, OC(O)N(R^{a1})₂, N(R^{a1})C(O)N(R^{a1})₂, wherein R^{a1} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
R^{b} is F, Cl, Br, I, NO₂, CN, R^{b1}, OR^{b1}, SR^{b1}, N(R^{b1})₂, SO₂R^{b1}, SO₂N(R^{b1})₂, C(O)R^{b1}; C(O)OR^{b1}, OC(O)R^{b1}; C(O)N(R^{b1})₂, N(R^{b1})C(O)R^{b1}, OC(O)N(R^{b1})₂, N(R^{b1})C(O)N(R^{b1})₂, wherein R^{b1} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
R^{c} is F, Cl, Br, I, NO₂, CN, R^{c1}, OR^{c1}, SR^{c1}, N(R^{c1})₂, SO₂R^{c1}, SO₂N(R^{c1})₂, C(O)R^{c1}; C(O)OR^{c1}, OC(O)R^{c1}; C(O)N(R^{c1})₂, N(R^{c1})C(O)R^{c1}, OC(O)N(R^{c1})₂, N(R^{c1})C(O)N(R^{c1})₂, wherein R^{c1} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
R^{d} is F, Cl, Br, I, NO₂, CN, R^{d1}, OR^{d1}, SR^{d1}, N(R^{d1})₂, SO₂R^{d1}, SO₂N(R^{d1})₂, C(O)R^{d1}; C(O)OR^{d1}, OC(O)R^{d1}; C(O)N(R^{d1})₂, N(R^{d1})C(O)R^{d1}, OC(O)N(R^{d1})₂, N(R^{d1})C(O)N(R^{d1})₂, wherein R^{d1} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
R^{e} is F, Cl, Br, I, NO₂, CN, R^{e1}, OR^{e1}, SR^{e1}, N(R^{e1})₂, SO₂R^{e1}, SO₂N(R^{e1})₂, C(O)R^{e1}; C(O)OR^{e1}, OC(O)R^{e1}; C(O)N(R^{e1})₂, N(R^{e1})C(O)R^{e1}, OC(O)N(R^{e1})₂, N(R^{e1})C(O)N(R^{e1})₂, wherein R^{e1} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
R^{f} is F, Cl, Br, I, NO₂, CN, R^{f1}, OR^{f1}, SR^{f1}, N(R^{f1})₂, SO₂R^{f1}, SO₂N(R^{f1})₂, C(O)R^{f1}; C(O)OR^{f1}, OC(O)R^{f1}; C(O)N(R^{f1})₂, N(R^{f1})C(O)R^{f1}, OC(O)N(R^{f1})₂, N(R^{f1})C(O)N(R^{f1})₂, wherein R^{f1} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
wherein any two or more of R¹, R², R³, R⁴, R^{a}, R^{b}, R^{c}, R^{d}, R^{e}, R^{f}, R^{z}, R^{zd}, R^{z1}, R^{z3}, R^{x1}, and R^{x2} may together form a ring.

129. The compound according to claim 128, wherein each of R¹, R², and R⁴ are hydrogen, and R³ is F, Cl, Br, I, NO₂, CN, SO₂R^{3a}, SO₂N(R^{3a})₂, C(O)R^{3a}; C(O)OR^{3a}, or C(O)N(R^{3a})₂.

130. The compound according to claim 128, wherein the [linker] has the formula: 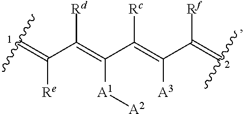
wherein:
A¹ is selected from C(R^{a1})₂, N(R^{a1}*)ₙₐ₁, O, S, —C(R^{a1})₂—C(R^{a1})₂—, —C(R^{a1})═C(R^{a1})—, —C(R^{a1})═N—;
R^{a1} is selected from F, Cl, Br, I, NO₂, CN, R^{a1$}, OR^{a1$}, N(R^{a1$})₂, SO₂R^{a1$}, SO₂N(R^{a1$})₂, C(O)R^{a1$}; C(O)OR^{a1$}, OC(O)R^{a1$}; wherein R^{a1$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
na1 is 1 or 2;
when na1 is 1 then R^{a1}* is hydrogen or C₁₋₈alkyl;
when na1 is 2 then R^{a1}* is in each case C₁₋₈alkyl;
A² is selected from null, C(R^{a2})₂, N(R^{a2}*)ₙₐ₂, O, S, —C(R^{a2})₂—C(R^{a2})₂—, —C(R^{a2})═C(R^{a2})—, —C(R^{a2})═N—;
R^{a2} is selected from F, Cl, Br, I, NO₂, CN, R^{a2$}, SR^{a2$}, SeR^{a2$}, OR^{a2$}, N(R^{a2$})₂, SO₂R^{a2$}, SO₂N(R^{a2$})₂, C(O)R^{a2$}; C(O)OR^{a2$}, OC(O)R^{a2$}; C(O)N(R^{a2$})₂, N(R^{a2$})C(O)R^{a2$}, OC(O)N(R^{a2$})₂, N(R^{a2$})C(O)N(R^{a2$})₂, wherein R^{a2$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
na2 is 1 or 2;
when na2 is 1 then R^{a2}* is hydrogen or C₁₋₈alkyl;
when na2 is 2 then R^{a2}* is in each case independently C₁₋₈alkyl;
A³ is selected from C(R^{a3})₂, N(R^{a3}*)ₙₐ₃, O, S, —C(R^{a3})₂—C(R^{a3})₂—, —C(R^{a3})═C(R^{a3})—, —C(R^{a3})═N—,
R^{a3} is selected from F, Cl, Br, I, NO₂, CN, R^{a3$}, OR^{a3$}, N(R^{a3$})₂, SO₂R^{a3$}, SO₂N(R^{a3$})₂, C(O)R^{a3$}; C(O)OR^{a3$}, OC(O)R^{a3$}; C(O)N(R^{a3$})₂, N(R^{a3$})C(O)R^{a3$}, OC(O)N(R^{a3$})₂, N(R^{a3$})C(O)N(R^{a3$})₂, wherein R^{a3$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
na3 is 1 or 2;
when na3 is 1 then R^{a3}* is hydrogen or C₁₋₈alkyl;
when na3 is 2 then R^{a3}* is in each case independently C₁₋₈alkyl.

131. The compound according to claim 130, wherein A² is CHR^{a2}, A¹ is CH₂ or CH₂CH₂, and A³ is CH₂ or CH₂CH₂.

132. The compound according to claim 130, wherein [linker] has the formula: 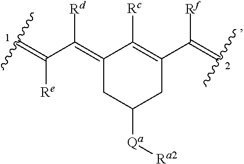
wherein Q^{a} is null, CH₂, CH₂CH₂, or CH₂CH₂CH₂, and R^{a2} is H, CH₃, CF₃, CH₂F, CHF₂, CH₂CH₃, CH(CH₃)₂, C(CH₃)₃, F, Cl, Br, OH, NH₂, SO₃H, PO₃H₂, COOH, OCH₃, OCH₂CH₃, OCH(CH₃)₂, OCF₃, OCH₂CF₃, NHCH₃, N(CH₃)₂, ϕ, CH₂CH₃, CH₂CF₃, CH₂CH₂F, CH₂CHF₂, CH(CH₃)₂, C(CH₃)₃, CH₂OH, CH₂NH₂, CH₂SO₃H, CH₂PO₃H₂, CH₂COOH, CH₂OCH₃, CH₂OCF₃, CH₂OCH₂CF₃, CH₂NHCH₃, CH₂N(CH₃)₂, CH₂ϕ, CH₂CH₂CH₃, CH₂CH₂CF₃, CH₂CH₂CH₂F, CH₂CH₂CHF₂, CH₂CH(CH₃)₂, CH₂C(CH₃)₃, CH₂CH₂OH, CH₂CH₂NH₂, CH₂CH₂SO₃H, CH₂CH₂PO₃H₂, CH₂CH₂COOH, CH₂CH₂OCH₃, CH₂CH₂OCF₃, CH₂CH₂OCH₂CF₃, CH₂CH₂NHCH₃, CH₂CH₂N(CH₃)₂, or CH₂CH₂ϕ,
wherein ϕ is an aryl or heteroaryl group.

133. The compound according to claim 128, wherein R^{d} and R^{f} are both hydrogen.

134. The compound according to claim 128, wherein R^{c} is F, Cl, Br, I, NO₂, CN, R^{c1}, SR^{c1}, OR^{c1}, or N(R^{c1})₂.

135. The compound according to claim 128, wherein R^{c} has the formula: 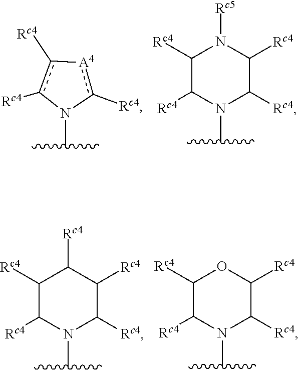
wherein A⁴ is selected from O, S, C(R^{c4})ₙₐ₄, N(R^{c5})ₙₐ₄;
na4 is 0, 1, or 2;
R^{c4} is case independently selected from F, Cl, Br, I, NO₂, CN, R^{c4$}, Q⁴-OR^{c4$}, Q⁴-N(R^{c4$})₂, Q⁴-SO₂R^{c4$}, Q⁴-SO₂N(R^{c4$})₂, Q⁴-C(O)R^{c4$}; Q⁴-C(O)OR^{c4$}, Q⁴-OC(O)R^{c4$}; Q⁴-C(O)N(R^{c4$})₂, Q⁴-N(R^{c4$})C(O)R^{c4$}, Q⁴-OC(O)N(R^{c4$})₂, Q⁴-N(R^{c4$})C(O)N(R^{c4$})₂, wherein Q⁴ is null or a C₁₋₄alkylene group, and R^{c4$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;
C^{c5} is in each case independently selected from R^{c5}, Q⁵-OR^{c5$}, Q⁵-N(R^{c5$})₂, Q⁵-SO₂R^{c5$}, Q⁵-SO₂N(R^{c5$})₂, Q⁵-C(O)R^{c5$}; Q⁵-C(O)OR^{c5$}, Q⁵-C(O)N(R^{c5$})₂, wherein Q⁵ is null or a C₁₋₄alkylene group, and R^{c5$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl; and
wherein any two of more of R^{c4} and R^{c5} can together form a ring.

136. The compound according to claim 135, wherein R^{c} is a group having the formula: 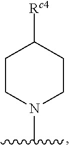
wherein
R^{c4} is F, Cl, Br, I, NO₂, CN, R^{c4$}, Q⁴-OR^{c4$}, Q⁴-N(R^{c4$})₂, Q⁴-SO₂R^{c4$}, Q⁴-SO₂N(R^{c4$})₂, Q⁴-C(O)R^{c4$}; Q⁴-C(O)OR^{c4$}, Q⁴-OC(O)R^{c4$}; Q⁴-C(O)N(R^{c4$})₂, Q⁴-N(R^{c4$})C(O)R^{c4$}, Q⁴-OC(O)N(R^{c4$})₂, Q⁴-N(R^{c4$})C(O)N(R^{c4$})₂;
Q⁴ is null or a C₁₋₄alkylene group; and
R^{c4$} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl.

137. The compound according to claim 128, wherein R^{c} is NR^{c1}R^{c2}, wherein
R^{c1} is selected from H, C₁₋₈alkyl, C₃₋₈cycloalkyl, C₁₋₈alkyl-OH, C₁₋₈alkyl-COOH, C₁₋₈alkyl-NH₂, C₁₋₈alkyl-NHCH₃, or C₁₋₈alkyl-N(CH₃)₂; and
R^{c2} is selected from H, C₁₋₈alkyl, C₃₋₈cycloalkyl, C₁₋₈alkyl-OH, C₁₋₈alkyl-COOH, C₁₋₈alkyl-NH₂, C₁₋₈alkyl-NHCH₃, or C₁₋₈alkyl-N(CH₃)₂,

138. The compound according to claim 128, wherein R^{c} is SR^{c1}, wherein R^{c1} is selected from H, CH₃, CH₂CH₃, CH(CH₃)₂, CH₂CH₂OH, CH₂COOH, CH₂CH₂COOH.

139. The compound of claim 128, wherein Z² is N—R^{zd}, and Z¹ is O, S, C(R^{z})₂ N—R^{z}, N═CR^{z1}, or C(CR^{z1})═C(CR^{z1}),

140. The compound according to claim 128, wherein Z¹ is C(CH₃)₂.

141. The compound according to claim 128, wherein both X¹ and X² are CN.

142. The compound according to claim 128, wherein either:
X¹ is Q²-CO₂H, Q²-CO₂C₁₋₃alkyl, Q²-CO₂C₁₋₃haloalkyl, Q²-SO₃H, Q²-SO₃C₁₋₃alkyl, Q²-SO₃C₁₋₃haloalkyl, Q²-SO₂C₁₋₃alkyl, CSO₂C₁₋₃haloalkyl, Q²-C(O)NHC₁₋₃alkyl, Q²-C(O)NHC₁₋₃haloalkyl, Q²-C(O)NH₂, Q²-C(O)NHC₁₋₃alkyl, Q²-C(O)NHC₁₋₃haloalkyl; Q² is null or —CH═CH—; and
X² is selected from Cl, F, CN, C₁₋₃haloalkyl; or
X² is Q²-CO₂H, Q²-CO₂C₁₋₃alkyl, Q²-CO₂C₁₋₃haloalkyl, Q²-SO₃H, Q²-SO₃C₁₋₃alkyl, Q²-SO₃C₁₋₃haloalkyl, Q²-SO₂C₁₋₃alkyl, CSO₂C₁₋₃haloalkyl, Q²-C(O)NHC₁₋₃alkyl, Q²-C(O)NHC₁₋₃haloalkyl, Q²-C(O)NH₂, Q²-C(O)NHC₁₋₃alkyl, Q²-C(O)NHC₁₋₃haloalkyl;
Q² is null or —CH═CH—; and
X¹ is selected from Cl, F, CN, C₁₋₃haloalkyl.

143. The compound of claim 128, wherein either:
X¹ is —CO₂H, —SO₃H, —CH═CH—CO₂H, —CH═CH—SO₃H, and X² is F, Cl, CN, or CF₃; or
X₂ is —CO₂H, —SO₃H, —CH═CH—CO₂H, —CH═CH—SO₃H, and X¹ is F, Cl, CN, or CF₃.

144. The compound according to claim 128, wherein [acceptor] has the formula: 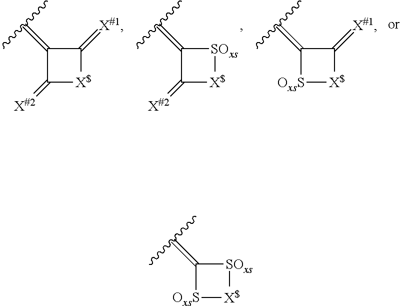
wherein xs is independently selected from 1 or 2;
X^{#1} and X^{#2} are independently selected from oxo, thioxo, imino, and CH₂, provided that both X^{#1} and X^{#2} are not each CH₂;
X^{$} is -ethylene-, -propylene-, -ethenyl-, O-methylene, —NR^{#3}-methylene-, O-ethylene, —NR^{#3}-ethylene-, —O-ethylene-O—, —NR^{#3}-ethylene-O—, —NR^{#3}-ethylene-NR^{#3}—, —O—C(═O)—O—, —NR^{#3}—C(═O)—O—, —NR^{#3}—C(═O)—NR^{#3}—, —O—C(═S)—O—, —NR^{#3}—C(═S)—O—, —NR^{#3}—C(═S)—NR^{#3}—, —O-propylene-, —NR^{#3}-propylene-, —O-propylene-O—, —NR^{#3}-propylene-O—, —NR^{#3}-propylene-NR^{#3}—, aryl, cycloalkyl, heteroaryl, or heterocyclyl,
wherein R^{#3} is in each case independently selected from H and C₁₋₄alkyl.
wherein said methylene, ethylene, ethenyl, propylene, propenyl, aryl, cycloalkyl, heteroaryl, and heterocyclyl may be substituted one or more times by halo, cyano, NH₂, COOH, CONH₂, —(CH₂CH₂O)ₒCH₃ (o=1-16), —(CH₂CH₂O)ₒH (o=1-16), C₁₋₃alkyl, C₁₋₃haloalkyl, C₁₋₃alkoxy, C₁₋₃haloalkoxy, C₁₋₃aminoalkyl, or C₁₋₃aminohaloalkyl.

145. A pharmaceutical composition, comprising the compound according to claim 128 and at least one pharmaceutically acceptable excipient.

146. A method of treating a proliferative disorder in a subject in need thereof, comprising administering to the subject the compound according to claim 128.

147. A method of imaging living tissue in a subject in need thereof, comprising administering to the subject the compound according to claim 128, and irradiating the compound.
